(19) United States
(12) Patent Application Publication
(10) Pub. No.: US 2014/0141049 A1
(43) Pub. Date: May 22, 2014

(54) METHOD AND COMPOSITION FOR IMPROVING SKIN CONDITIONS COMPRISING HUMAN PLACENTAL LACTOGEN AS AN ACTIVE INGREDIENT

(71) Applicant: Regeron, Inc., Chuncheon Gangwon-Do (KR)

(72) Inventors: Dahlkyun Oh, Chuncheon (KR); Kyunyoung Lee, Chuncheon (KR)

(21) Appl. No.: 14/072,787

(22) Filed: Nov. 5, 2013

Related U.S. Application Data
(60) Provisional application No. 61/722,781, filed on Nov. 5, 2012.

(51) Int. Cl. A61K 38/22; A61Q 19/00; A61K 8/64; A61K 9/127; A61K 8/14

(52) U.S. Cl. CPC A61K 38/22; A61K 9/127; A61K 8/14; A61K 8/64; A61Q 19/00
USPC 424/401; 514/9.7; 424/450

(57) ABSTRACT

Disclosed herein is a skin condition-improving composition for topical application to the skin, comprising human placental lactogen (hPL) as an active ingredient. The disclosed method and composition exhibit various skin-conditioning effects, such as preventing and/or reducing atopic dermatitis, ultraviolet-light-caused skin damage, wrinkles, age spots, skin pigmentation, acne, itching, xerosis, and skin aging; and such as improving skin elasticity and moisturization.

### CROSS-REFERENCE TO RELATED APPLICATIONS

This application is claims the benefit of and priority to U.S. Provisional Patent Application Ser. No. 61/722,781, filed on Nov. 5, 2012, the contents of which are hereby incorporated by reference herein in their entirety.

### FIELD OF THE INVENTION

The present invention relates to skin condition-improving methods and compositions for topical application to the intact skin, which comprises human placental lactogen as an active ingredient.

### BACKGROUND OF THE INVENTION

Traditionally, proteins were not considered as qualified candidates for cosmetic active ingredients mainly due to: 1) their intrinsic labile nature in an aqueous solution; and 2) their formidable size barriers in reaching viable skin layers.

Proteins are in general hydrophilic or occasionally hydrophobic macromolecules composed of more than 20 amino acids with approximate molecular weight (m.w.) of 2000 dalton(2 kd) or higher. Oligopeptides, on the other hand, normally consists of less than 20 amino acids. In the cosmetic field, peptides normally refer to oligopeptides, often composed of fewer than 10 amino acids. Traditionally, macromolecules with m.w. of more than 500 dalton were considered difficult to pass through the skin epidermis due to its keratin barrier (Bos J. D. et al., Experimental Dermatology, 2000, 9(3), 165-169). Even with the help of chemical penetration enhancers, macromolecules with m.w. more than 2000 dalton were considered practically implausible to permeate through the skin epidermis. Therefore, when peptides are developed as cosmetic ingredients, an oligopeptide consisting of less than 10 amino acids (m.w. roughly about 1100 dalton) are adopted, if possible, to enable it to reach the skin dermis where most esthetically meaningful outcomes are firmly believed to be induced by functional cosmetic actives. Given these notions, it is not sensible to use a protein as large as hPL (human placental lactogen) with 191 amino acids (m.w., ˜22000 dalton) as an active cosmetic ingredient on the intact skin. What was more unexpected was, despite overwhelming skepticism, the experimental outcome of topical application of liposome-encapsulated hPL on the intact skin in vivo, both in human and mouse, that clearly showed its efficacies on the skin as diverse as acne alleviation, sunlight-caused dark spots removal in addition to skin tone improvement in human applications and UV-induced wrinkle removal in mouse experiments. An attempt to encompass and rationalize these two seemingly contradicting experimental observations—one, impermeability of the skin to a macromolecule like hPL and the other, experimental in vivo efficacies of hPL on the intact skin—resulted in the present invention.

Human placental lactogen (hPL) is a hydrophilic polypeptide composed of 191 amono acids and has approximate m.w. of 22 kd. hPL has two pairs of disulfide bonds and extensive α-helical structural motifs. Together they confer compactness and sturdiness on hPL molecules. However, although hPL can be considered as a stable protein by the norms of the protein world, it is of course subject to various physical, chemical, and biological deteriorations as most other proteins are. It needs protection from oxidative damage, conformational changes, enzymatic degradation, aggregation, precipitation, etc. Moreover, though hPL may be stored more than 2 years through freeze-drying with the help of stabilizing disaccharide like lactose or sucrose, once it is dissolved in an aqueous solution, hPL becomes labile and susceptible to a variety of assaults just mentioned above; thereby limiting its application as a cosmetic ingredient, let alone its efficacies on the intact skin. Thus, it is imperative to first find a protective carrier for hPL that enable hPL to reach a target tissue layer and at the same time can shield hPL from protease attacks and adverse conditions in the way.

The weakest point of cosmetic ingredient with a protein as an active ingredient is its labile nature such as its intrinsic propensity for denaturation, degradation, or aggregation in a solution state. Thus, if hPL can be used as a cosmetic ingredient, it should overcome minimum stability requirements set for a cosmetic product. hPL is prone to aggregation and degradation in solution, but much of it can be overcome by encapsulating hPL inside a liposome.

It is well known that hydrophilic macromolecular proteins like hPL can not penetrate the skin epidermis by itself due to hydrophobic nature of the epidermal keratin layer. Previous attempts to deliver proteins deeper into the dermis of the skin are scarce and grossly unsuccessful, mainly due to impermeability of the skin epidermal barrier to hydrophilic proteins. Among these inefficient attempts were some that even used liposomes as protein carriers to expedite protein penetration into the skin dermis. Conclusions from those experiments can be summarized as follows: first, protein translocation through the epidermis is a very inefficient process even when the receiver side of the skin tissue is in direct contact with an aqueous phase; second, it's not a practical delivery route for therapeutic proteins that need systemic distribution (du Plessis J et al., Antiviral Res. 1992, 18(3-4), 259-265). As main objectives of those previous experiments were to monitor the possibility of systemic protein delivery through the skin, most of the evaluations were made using in vitro Franz diffusion cell systems based on the amount of proteins that physically translocated the whole span of the skin depth under examination. Thus, in retrospect, these systems could have missed the in vivo biological effects caused by proteins in transit or entrapped inside the respective skin. Nonetheless one of those early experiments hinted at forthcoming of protein cosmetic ingredients; for example, although penetration of liposomal gamma-interferon through the skin in vitro was very inefficient at best, it was able to elicit biological response in the form of a secondary effector protein expression in vivo.

Recent studies on the topical delivery of macromolecules recognized the importance of hair follicular or transfollicular route of delivery. In this mode of macromolecular delivery, liposomes turn out to be one of the best carriers in which a target molecule can be transported to the pocket of the hair follicle. It seems dependent on the size, surface charge, flexibility, and, possibly, composition of the liposomes carrying target molecules. As the average diameter of the hPL-encapsulating liposome can be controlled to around 200 nm and a particle with up to 5 um-diameter has been observed to enter the hair follicle, taken together, it is not too difficult to imagine that liposome-encapsulated hPL certainly can enter the skin hair follicles and interact with the outermost viable cells constituting the hair follicles. However, it is not likely that macromolecular hPL penetrates the epidermal layer of the hair follicle wall into the dermis, considering a plethora of previous reports attesting otherwise.

One significant conceptual addition to the follicular delivery of hPL came from recent studies focused on elucidating the location of the skin stem cells. Traditionally, epidermal basal layer cells were thought to be the stem cells for the epidermal skin. Though the epidermal basal layer seemed to contain its own pool of stem cells, specifically termed “interfollicular stem cells” that give rise to skin epidermal layer cells, it turned out that a small bulge region just under the sebaceous gland in the hair follicle contains the stem cells, dubbed as “bulge stem cells”, that can supply all kinds of skin cells of the epidermis including hair follicles. More specifically, the bulge stem cells are the cells that divides slowly and steadily to give progenitor cells termed “transiently amplifying progenitor cells” that become fast dividing cells possessing a limited proliferative capacity as they migrate toward their presumed destinations and progressively differentiate into epidermal cells, sebaceous gland cells, or hair follicle matrix cells. In other words, the hair follicles are one of the key skin constituents where cosmetic treatments for any beneficial change in the epidermal skin biology should focus on.

For delivery of proteins through skin hair follicles on skin, either a delivery system in the form of a liposome or a lipid composite comprising lipids such as fatty acids has been reported to be favorable (Meidan V M et al., Int J Pharm. 2005, 306(1-2), 1-14; Lauer A. C. et al., Advanced drug delivery reviews v.18 no.3, pp.311-324, 1996; Wosickaa H. et al., Journal of Dermatological Science 2010, 57:83-89). In addition, although efficiencies turned out to be much lower, an aqueous solution containing an organic solvent such as ethanol or an aqueous solution containing a polymer such as polyethylene glycol, has also been tested as a facilitating medium for delivery through hair follicles. With respect to efficiencies of delivering proteins through the skin using liposomes, a general principle has not been established yet, because cases of liposomal protein deliveries have been scarce and even in those rare cases, efficiencies of protein deliveries varied widely depending on an empirical choice of a target protein and the nature of the liposome used. When a protein in a liposome-encapsulated form is to be delivered into skin hair follicles, the following factors at minimum and their complex interactions seem to determine the follicular delivery efficiency: 1) the liposome carrying the protein—overall size and outer and inner surface charges of the liposome in aqueous solution and characteristics of the amphiphilic components constituting the liposome; 2) the cargo protein encapsulated by the liposome; 3) the infundibulum and the inner sheath environments of the hair follicle through which the liposome makes inroads into. Thus as a whole, these three factors and their complex interactions seem to determine follicular delivery efficiencies of the liposomes containing the proteins in question through empirical formulations rather than by a general guiding principle at the moment.

One of the prevailing preoccupations in the cosmetic field, often implicit, is the notion that “whatever cosmetic ingredient biologically effective to the skin entails some irritation; the more, the severer.” While most, if not all, of the well-known cosmetic active ingredients, including retinoids and AHA's, have fallen within the boundary of this quotation, hPL definitely turned out not to be one of those despite much unfounded concern. In fact, no irritation whatsoever, if properly processed, is the distinctive hallmark of liposome-encapsulated hPL applied on the intact skin. The rationale behind this observation can be recapitulated as follows: 1) hPL is too large a molecule to pass through the epidermal layer of the hair follicle; 2) hPL molecules exposed to inside the hair follicle upon liposome disintegration are subject to rapid protease degradation due to the environment rich in degradative enzymes; 3) the cells having chances of direct interaction with hPL, that is, viable cell layers of epidermis, will most likely undergo apoptosis and naturally be shed from the skin in about several weeks, thereby leaving no long-term potential aberration to the skin; 4) the concentration of liposome-encapsulated hPL deliverable to the skin, compared to normal endogenous hPL level in the circulating blood, is such that it won't amount to any physiologically significant systemic entity to the body except the skin under the direct application of the liposome-encapsulated hPL. Therefore, there is practically no risk of overdosing hPL to the intact skin.

So far, liposome-encapsulated hPL has been formulated into toner (essence), cream, serum, and gel forms and been made to maintain its integrity in terms of the biological activity. Once liposome-encapsulated hPL has been properly formulated, those formulations sometimes seem to turn out more stable than the liposome-encapsulated hPL itself. This is probably due to a “cage effect” in which the water phase containing liposome-encapsulated hPL is divided into tiny droplets and individually firmly surrounded by a hydrophobic water repellent wall, providing safe harbor for liposome-encapsulated hPL particles by limiting their turbulent encounters with one another, hence stabilizing the liposomal shield surrounding hPL. By a similar principle, nanoliposomes surrounded by the water-soluble polymers can be stabilized due largely to the swelling nature of the polymers in aqueous solution and their accommodating nanoliposomes into sequestration inside polymeric 3-dimensional criss-crossed structures. This extra-stabilizing effect is pronounced and illustrated especially in the fact that some of these formulations kept at the temperature as high as 55° C. can stably maintain hPL bioactivity for a prolonged period of time while the liposome-encapsulated hPL itself fails to do so. Thus, it is even more important to take proper precaution not to break a liposomal shield during the formulating processes. Some rules of thumb avoiding a liposome breakage are: 1) keep the mixing temperature above 4° C. but as cool as possible, and if exposure to high temperature is not avoidable, run high temperature mixing operations involving liposome-encapsulated hPL as short and as late as possible; 2) do not put liposome-encapsulated hPL under pH lower than 5 or higher than 10 under any circumstances as it can cause precipitation or dissolution of liposomes, respectively; 3) keep liposome-encapsulated hPL away from direct contact with organic solvents that can destabilize liposomes.

To summarize, proteins were not traditionally considered proper candidates for active cosmetic ingredients mainly due to their intrinsic labile nature in aqueous solution on one hand and our conceptual difficulty in delivering their formidable sizes to the viable skin layers on the other. However, those barriers can be overcome. Should a protein be a cosmetic active ingredient, it needs to satisfy the following prerequisites: first, it can be formulated to stay stable at room temperature for a prolonged period of time; second, it should be innocuous to the skin and readily available for interaction with the viable cells in the hair follicle; third, the interacting cells in the hair follicle should have receptors for the protein of interest on their surfaces; fourth, the ligand-receptor interaction should lead to long-term beneficial effects on the skin. Proteins can, if appropriately chosen and formulated, be the most efficacious yet safest cosmetic ingredients available and that hPL is definitely one of those proteins so far known to the cosmetic field.

Human placental lactogen (hPL) is a placental peptide hormone and secreted from syneytio-trophoblast only during pregnancy for supporting the fetal growth. It is a dimeric protein consisting of 191 amino acids and two disulfide bonds. It is known to stimulate amino acid transport, DNA synthesis, and insulin-like growth factor production in isolated fetal fibroblasts and myoblasts (Fowlkes J et al., Endocrinology. 132 (6):2477-2483, (1993)).

Placental lactogen, growth hormone and prolactin are members of the growth hormone subfamily of an extensive cytokine superfamily of growth factors and their receptors that share many of the same general structure-function relationships in expressing their biological activities. (Kossiakoff A A, Adv Protein Chem. 68:147-69 (2004)). However, unlike Placental lactogen, growth hormone regulates growth and development in the postnatal period but lacks somatotropic activity in the fetus, suggesting that PL may function as a “fetal GH.” (Fowlkes J et al., Pediatric research 32(2): 200-203 (1992); Gertler A., J Mammary Gland Biol Neoplasia. 2(1):69-80, (1997)). These hormones and receptors are thought to have arisen as a result of gene duplication and subsequent divergence early in vertebrate evolution. Mammalian growth hormone and prolactin show a slow basal evolutionary rate of change, but with episodes of accelerated evolution. Placental lactogen has probably evolved independently on three occasions, from prolactin in rodents and ruminants and from growth hormone in man. (Forsyth I A et al., J Mammary Gland Biol Neoplasia., 7(3):291-312, (2002)). Prolactin, growth hormone and placental lactogen, along with two non-classical members of the family, proliferin and proliferin-related protein, can act both as circulating hormones and as paracrine/autocrine factors to either stimulate or inhibit various stages of the formation and remodeling of new blood vessels, including endothelial cell proliferation, migration, protease production and apoptosis (Corbacho A et al., J. Endocrinol. 173(2):219-238 (2002).

However, although the structure and functions of human placental lactogen (hPL) seem to be similar to those of human growth hormone (hGH) in some aspects, its ability to promote energy supply to the fetus is at least 10 times higher than hGH. Results of bioassays have revealed some functional similarities between hPL and prolactin (PRL), but it is not clear what the exact role of hPL is in a nursing mother. Human placental lactogen is also known to have anti-insulin activity as it influences the metabolic system of a pregnant mother through increasing the blood glucose level by suppressing the insulin sensitivity and decreasing maternal consumption of glucose so that more glucose can be supplied to the fetus. It also stimulates the production of ketone from free fatty acids which can easily permeate through placental membrane hence readily provided to the baby. (Guyton et al., Textbook of Medical Physiology (11 ed.). pp. 1033 (2005)).

hPL is only present during pregnancy with maternal serum levels rising in relation to the growth of the fetus and placenta. Maximum levels are reached near term, typically to 5-7 mg/ml. Higher levels are noted in patients with multiple gestation.

Peptides of amino acid sequences identical with or similar to N-terminal 28 amino acids of mature hPL have been identified and suggested as a pharmaceutical composition for treatment of thrombosis-related diseases (see, e.g., U.S. Patent Publication No. 2013035289), or for treatment of disorders such as autoimmune diseases, inflammatory diseases, and transplant rejection, which are associated with increased expression of interferon-gamma-stimulated major histocompatibility complex antigens (U.S. Patent Publication No. 2002/0032154). Bovine placental lactogen has been recombinantly produced to enhance the growth of farm fishes (U.S. Pat. No. 6,136,562, U.S. Pat. No. 5,010,011).

### SUMMARY OF THE INVENTION

The present invention provides methods and compositions comprising human placental lactogen (hPL) as the active ingredient, which are capable of improving various skin conditions.

The invention further provides that the methods and compositions described above are using hPL encapsulated in liposome to promote its delivery to the skin hair follicles and to maintain the prolonged activity of hPL when applied topically.

The invention also encompasses the methods of improving the skin conditions, such as atopic dermatitis, contact dermatitis, skin damages caused by ultraviolet-light exposure, photoaging, UV or sunburn-induced pigmentation, wrinkles, age spots, acne, dryness, itching, actinic keratosis, eczema, psoriasis, sunburn, skin elasticity problems, and skin aging; and improving skin elasticity and moisturization by topical application of the compositions.

Thus, in one aspect, the invention provides a method for improving skin conditions of a human, which comprises topically administering to the skin of a human a composition comprising an effective amount of human placental lactogen as an active ingredient, wherein the skin condition is selected from the group consisting of atopic dermatitis, wrinkles, age spots, acne, itching, poor skin elasticity, skin aging and poor skin moisture; wherein the composition is applied to a normal skin surface that is not in direct contact with blood (e.g., a skin surface that is not in contact with circulating blood).

In another aspect, the invention provides a method for improving skin conditions of a human, which comprises topically administering to the skin of a human a composition comprising an effective amount of human placental lactogen as an active ingredient, wherein the skin condition is selected from the group consisting of atopic dermatitis, wrinkles, age spots, skin pigmentation, acne, itching, xerosis, poor skin elasticity, skin aging and poor skin moisture; wherein the composition is applied to a normal skin surface that is not in direct contact with blood.

In another aspect, the invention provides a method for improving skin conditions of a human, which comprises topically administering to the skin of human a composition comprising an effective amount of human placental lactogen as an active ingredient, wherein the skin condition is selected from the group consisting of atopic dermatitis, wrinkles, age spots, acne, itching, poor skin elasticity, poor hair growth, skin aging and poor skin moisture; wherein the composition is applied to a normal skin surface that is not in direct contact with blood.

In certain embodiments, the skin surface is unbroken, i.e., not wounded, ulcerated, or otherwise compromised.

In certain embodiments, the human placental lactogen is encapsulated into a liposome. In certain embodiments, the liposome is a nanoliposome. In certain embodiments, the nanoliposome has a particle size of 50-250 nm. In certain embodiments, the nanoliposome has a small unilamellar vesicle structure.

In certain embodiments, the unencapsulated human placental lactogen encapsulated into the nanoliposome has the activity of 90-100% of an unencapsulated human placental lactogen.

In certain embodiments, the composition is a cosmetic or pharmaceutical composition.

In certain embodiments, the invention provides a method for improving skin conditions of a human, which comprises topically administering to the skin of human a composition comprising an effective amount of human placental lactogen as an active ingredient, wherein the skin condition is selected from the group consisting of atopic dermatitis, contact dermatitis, skin damages caused by ultraviolet-light exposure, photoaging, UV or sunburn-induced pigmentation, wrinkles, age spots, acne, dryness, itching, actinic keratosis, eczema, psoriasis, sunburn, skin elasticity problems, and skin aging.

In certain embodiments, the invention provides a method for improving skin conditions of a human, which comprises topically administering to the skin of human a composition comprising an effective amount of human placental lactogen as an active ingredient, wherein the skin condition is selected from the group consisting of atopic dermatitis, contact dermatitis, skin damages caused by ultraviolet-light exposure, photoaging, UV or sunburn-induced pigmentation, wrinkles, age spots, acne, dryness, itching, hair-loss, actinic keratosis, eczema, psoriasis, sunburn, skin elasticity problems, and skin aging.

In another aspect, the invention provides a topical composition comprising an effective amount of human placental lactogen as an active ingredient. In certain embodiments, the human placental lactogen is encapsulated into a liposome. In certain embodiments, the liposome is a nanoliposome. In certain embodiments, the nanoliposome has a particle size of 50-250 nm. In certain embodiments, the nanoliposome has a small unilamellar vesicle structure.

In certain embodiments, the unencapsulated human placental lactogen encapsulated into the nanoliposome has the activity of 90-100% of an unencapsulated human placental lactogen.

### BRIEF DESCRIPTION OF THE FIGURES

FIG. 1 shows the amino acid sequence of human placental lactogen (hPL) consisting of the signal peptide (amino acid residues 1-25), mature hPL polypeptide (26-216) which was used as the active ingredient of the skin-improving composition.

FIG. 2 is the nucleic acid sequence of human placental lactogen, which encodes the hPL protein. The nucleic acid residues of hPL from 1 to 8 shown in black font represents the part from exon 1, the residues from 9 to 168 in blue font is from exon 2, the residues from 169 to 288 in red font is from exon 3, the residues from 289 to 453 in green font is from exon 4, the residues from 456 to 651 in purple font is from exon 5. The underlined part (base pairs from 1-75) represents the signal peptide.

FIG. 3 describes cultivation results of the cell line RZ4500 transformed by the expression vector construct (pUC-narK-Met-hPL). Cell culture was performed in the 7 liter capacity fermentation vessel to obtain the recombinant hPL. Also presented in this figure is the schematic diagram of the expression vector pUC-narK-Met-hPL.

FIG. 4 represents the results of gel filtration chromatography for the purification of recombinant hPL

FIG. 5 shows the improvement in the skin wrinkle condition of nude mice by topical application of hPL encapsulated in liposome (Lipo-hPL)

FIG. 6 is the graphical summary of increase in the skin elasticity when Lipo-hPL was topically applied.

FIG. 7 is the graphical summary of the effects of Lipo-hPL on TEWL (trans-epidermal water loss) showing suppression of skin dryness by topical application of Lipo-hPL

FIG. 8 shows H&E stained tissue pictures to demonstrate the improvement of the skin damages due to the exposure to ultraviolet light by topical application of Lipo-hPL

FIG. 9 shows changes in the skin condition of Nc/Nga mouse with atopic dermatitis after the short term topical application of Lipo-hPL.

FIG. 10 is the graphical summary of changes in the skin damage condition of Nc/Nga mouse with atopic dermatitis after the short term topical application of Lipo-hPL

FIG. 11 shows changes in the skin condition of Nc/Nga mouse with atopic dermatitis after the long term topical application of Lipo-hPL

FIG. 12 is the graphical summary of changes in the skin damage condition of Nc/Nga mouse with atopic dermatitis after the long term topical application of Lipo-hPL

FIG. 13 describes the changes in the blood IgE concentration of Nc/Nga mouse with atopic dermatitis after the short term topical application of Lipo-hPL

FIG. 14 demonstrates the H&E stained results of changes in the skin tissue of Nc/Nga mouse with atopic dermatitis after the short term topical application of Lipo-hPL

FIG. 15 demonstrates the H&E stained results of changes in the skin tissue of Nc/Nga mouse with atopic dermatitis after the long term topical application of Lipo-hPL

FIG. 16 shows the graphical summary of changes in the skin thickness of Nc/Nga mouse with atopic dermatitis after the long term topical application of Lipo-hPL

FIG. 17 shows the effect of Lipo-hPL in reducing age spots

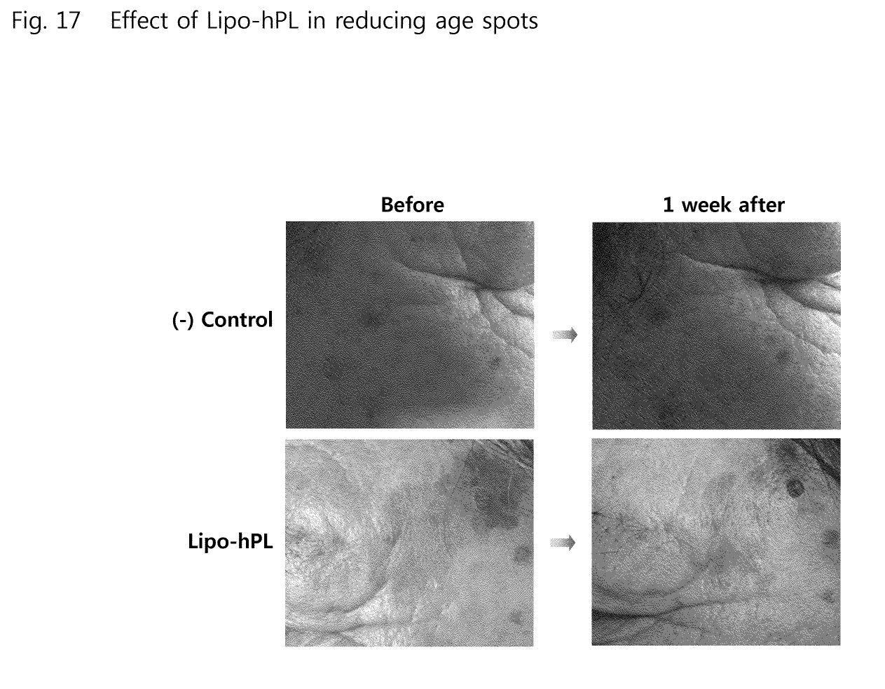

FIG. 18 shows the anti-acne effect of Lipo-hPL

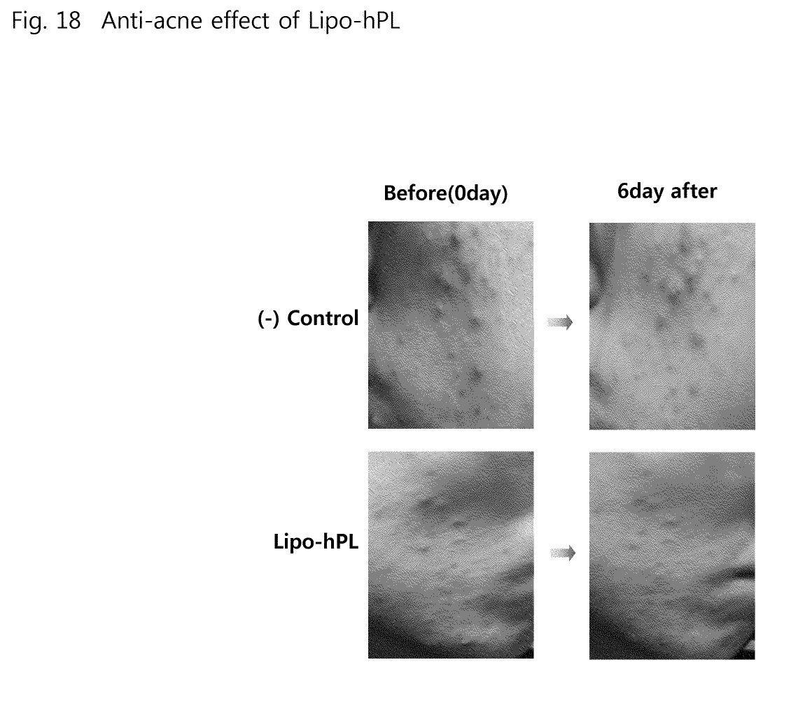

FIG. 19 shows the epidermal changes of artificial skin treated with Lipo-hPL

FIG. 20 shows the effect of Lipo-hPL on DNFB (1-fluoro-2,4-dinitrobenzene) treatment-induced ear thickness in Nc/Nga mice

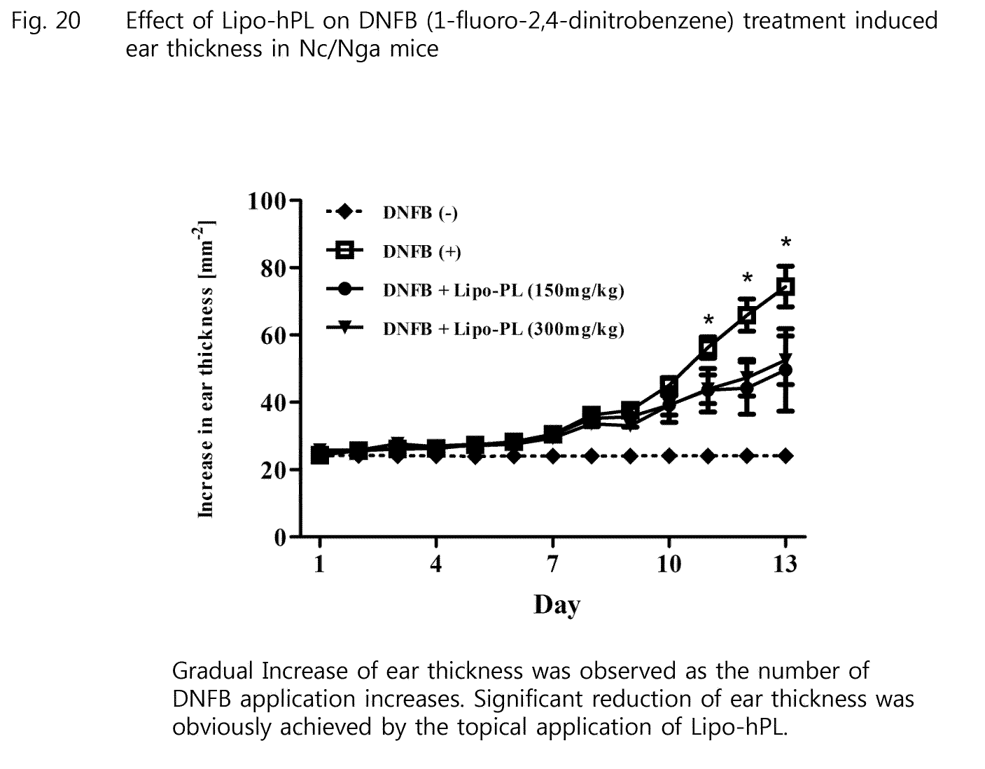

FIG. 21 shows the effects of topical Lipo-hPL application on skin damages induced by the topical DNFB treatment and the valuation of DNFB-induced skin damage (Clinical skin severity score)

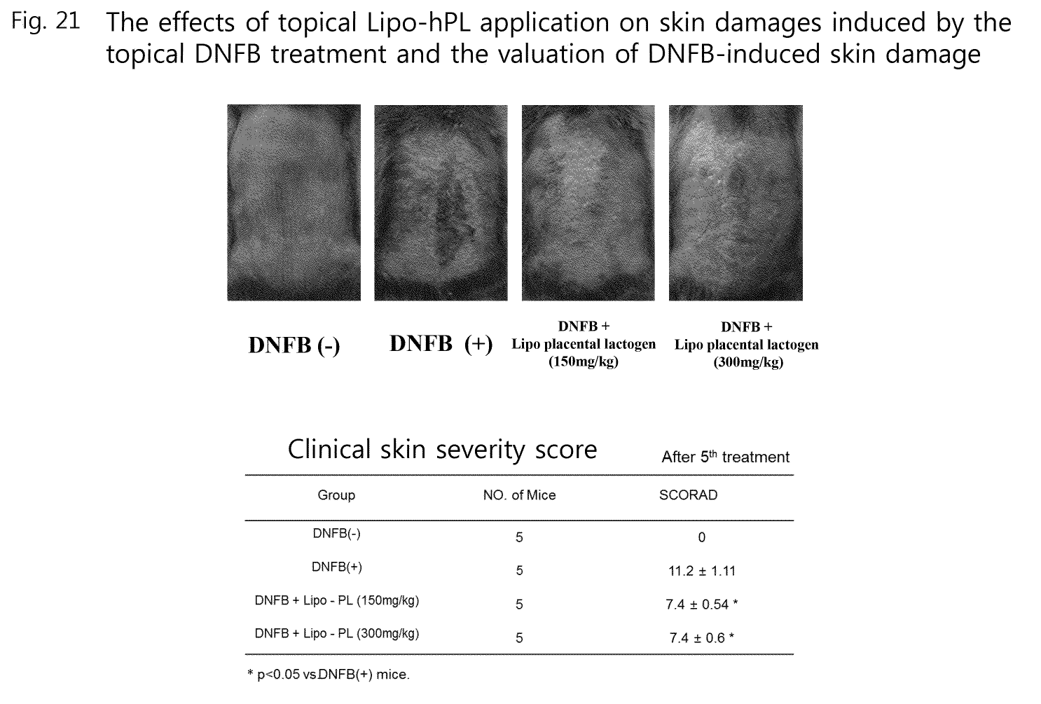

FIG. 22 shows the Effects of Lipo-hPL on: 1) the production of IgE in the blood; and 2) production of IL-4 and IFN-γ by CD4+ T cells in DNFB-induced Nc/Nga mice

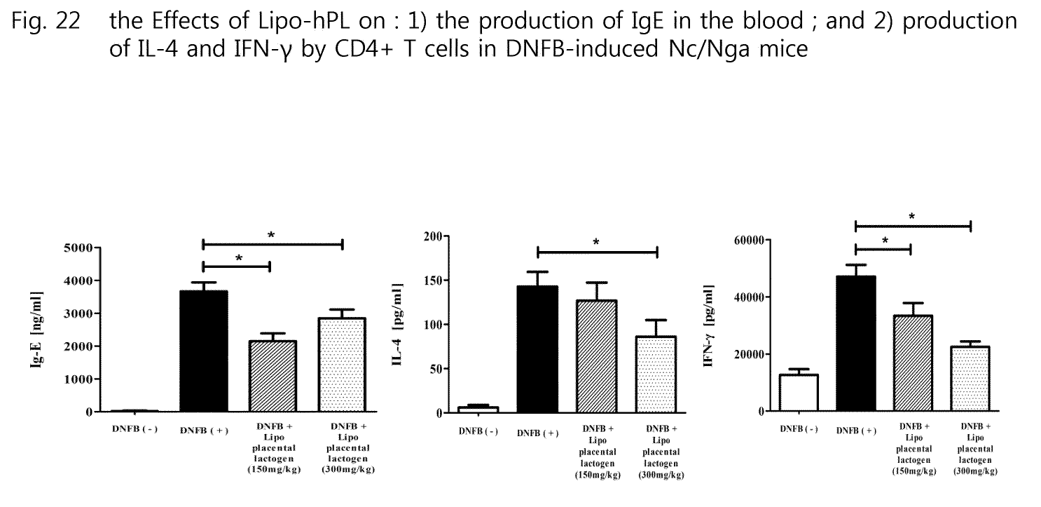

FIG. 23 shows the effects of topical application of Lipo-hPL on changes in the H&E-stained skin tissue of DNFB-induced atopic dermatitis in Nc/Nga mice

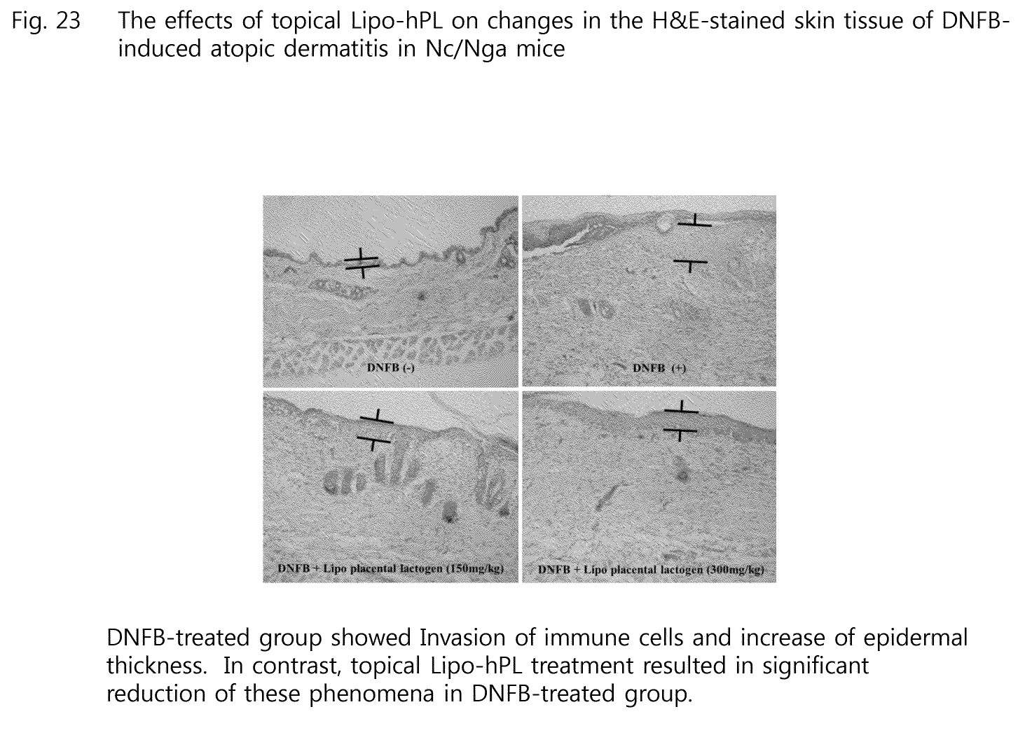

FIG. 24 shows the effects of topical application of Lipo-hPL on invasion of mast cells in the toluidine blue-stained skin tissue of DNFB-induced atopic dermatitis Nc/Nga mice

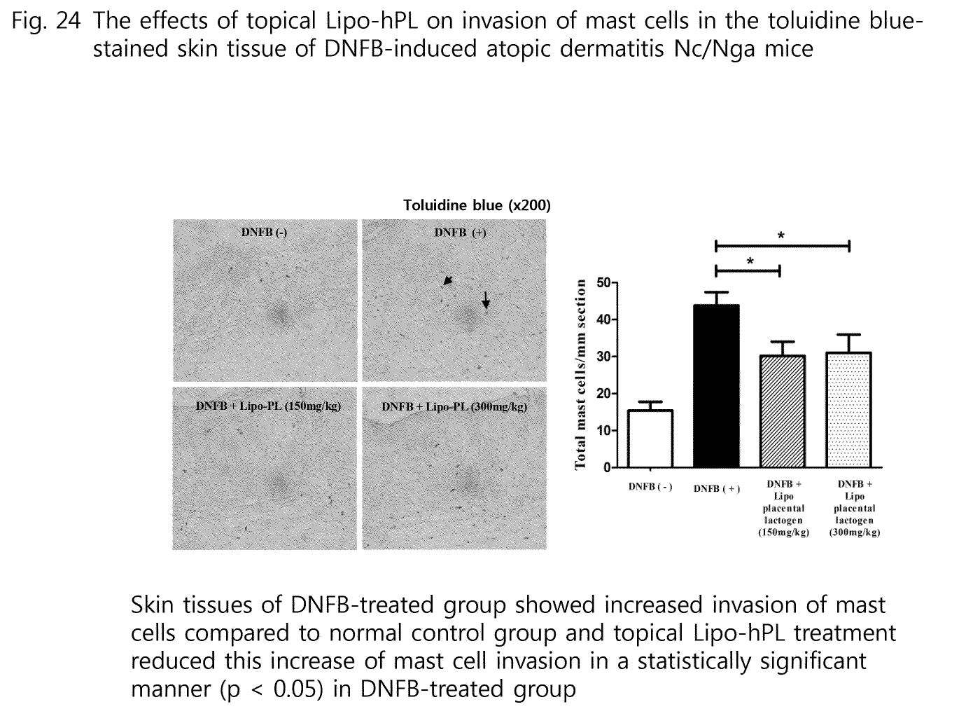

FIG. 25 shows the effects of topical Lipo-hPL application on changes of Filaggrin expression in skin tissues of Nc/Nga with DNFB-induced atopic dermatitis

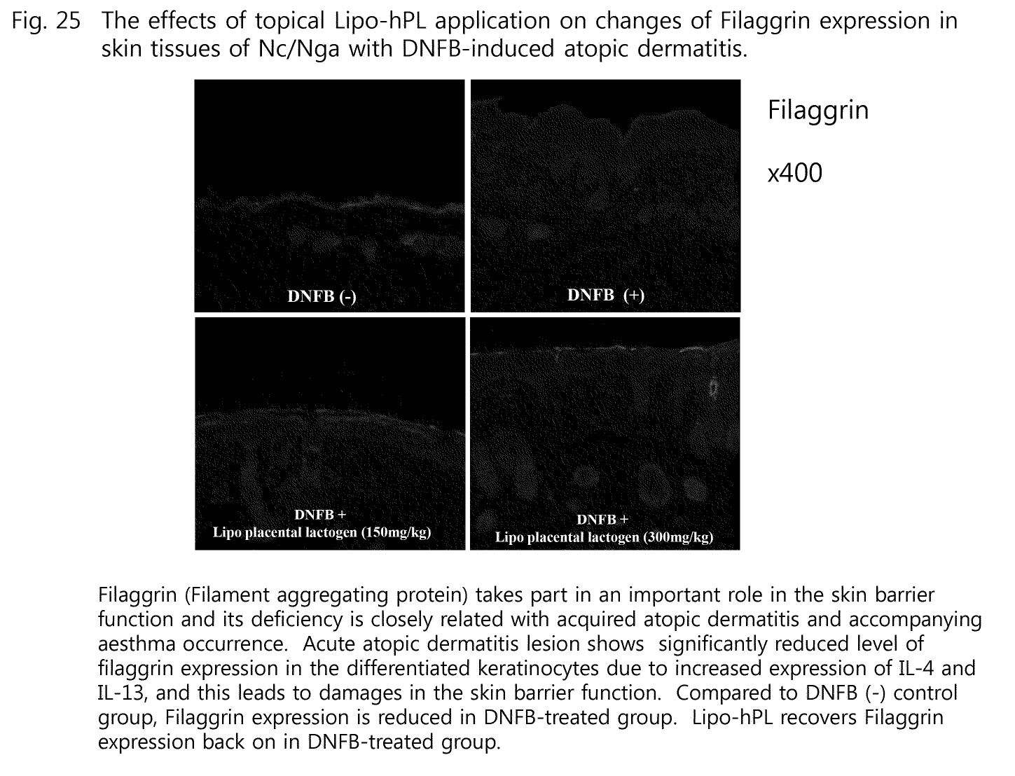

FIG. 26 shows the effects of topical application of Lipo-hPL on invasion of CD4+ T-cells in skin tissue of DNFB-induced atopic dermatitis Nc/Nga mice

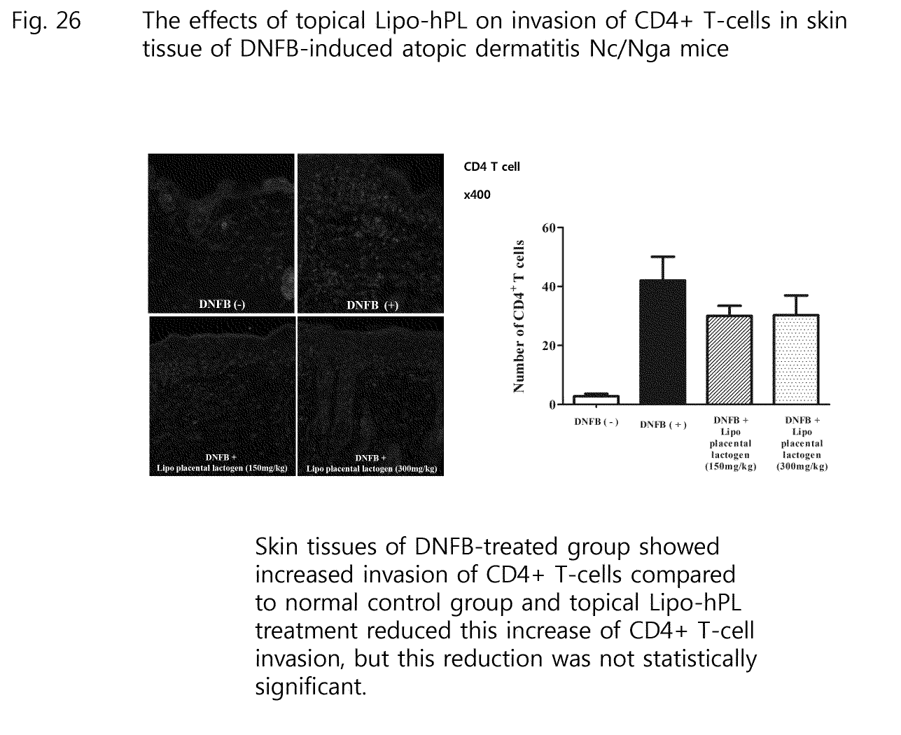

FIG. 27 shows the effects of topical Lipo-hPL on invasion of CD8+ T-cells in skin tissue of DNFB-induced atopic dermatitis Nc/Nga mice

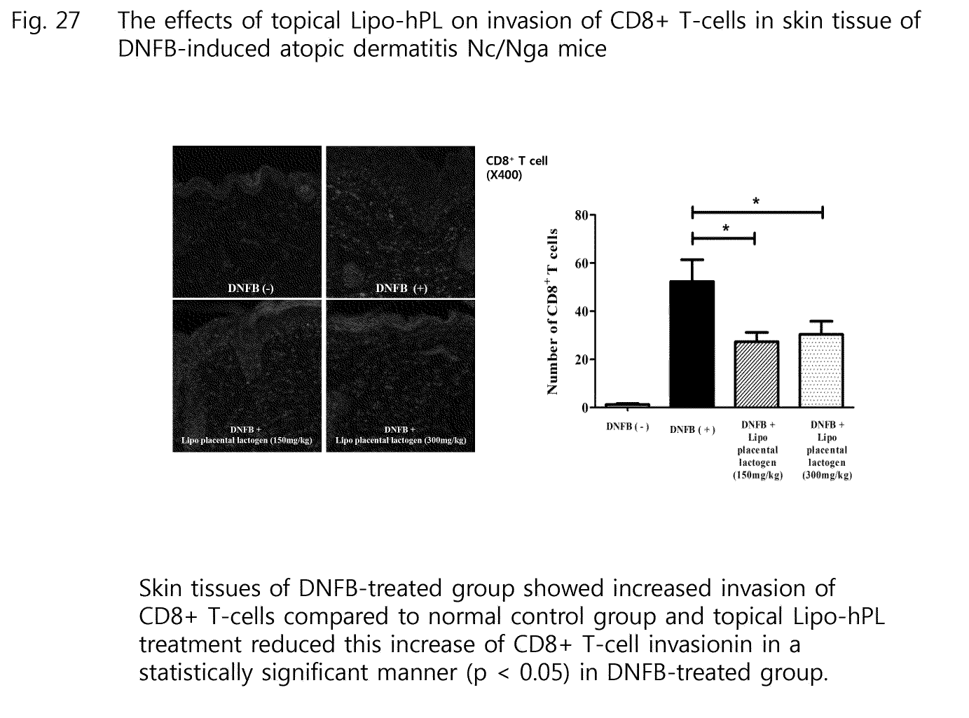

FIG. 28 shows the clinical test results on the improvement of skin hydration by topical Lipo-hPL

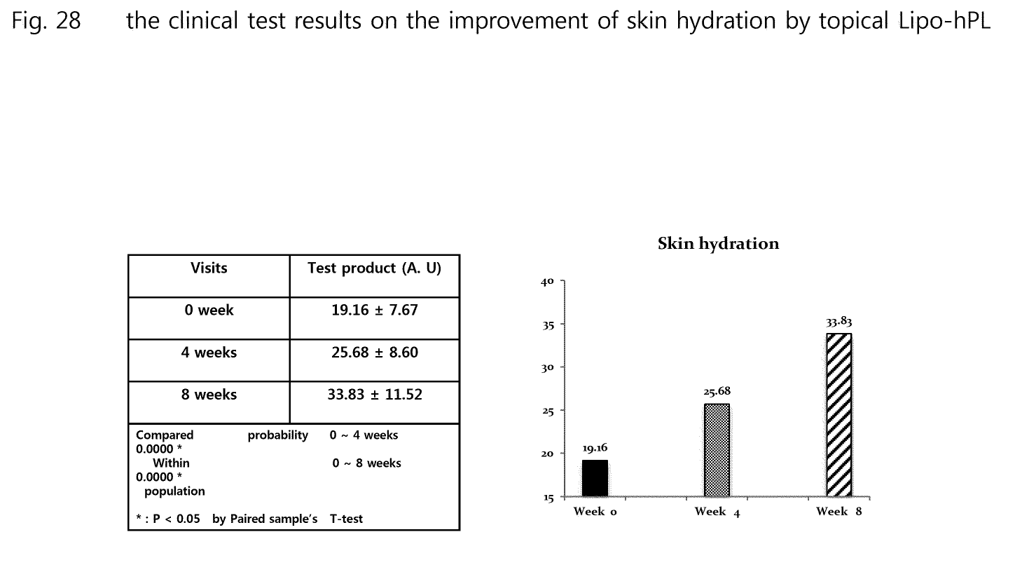

FIG. 29 shows the clinical test results on the improvement of TEWL by topical Lipo-hPL

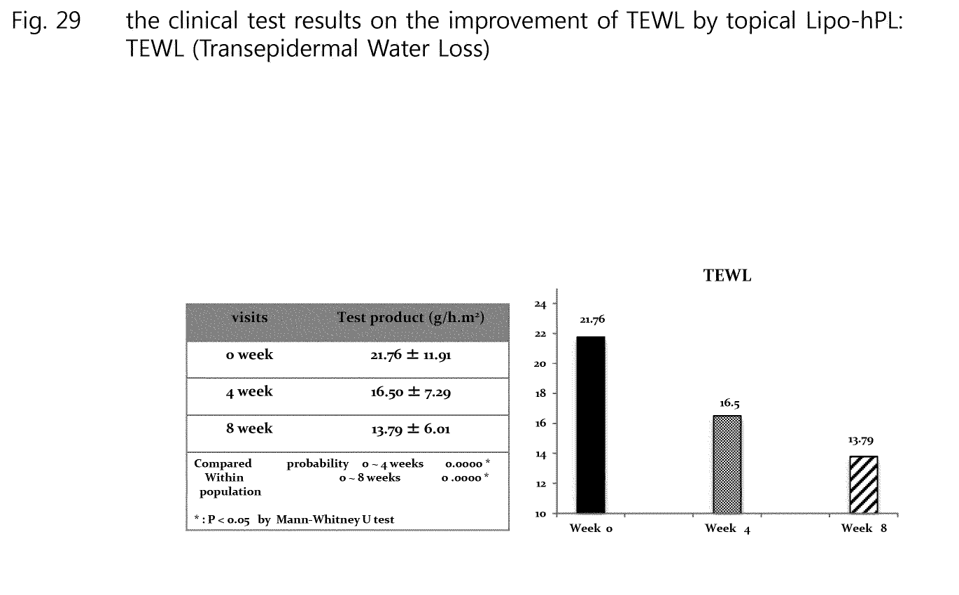

FIG. 30 shows the clinical test results on the change of skin temperature after application of topical Lipo-hPL

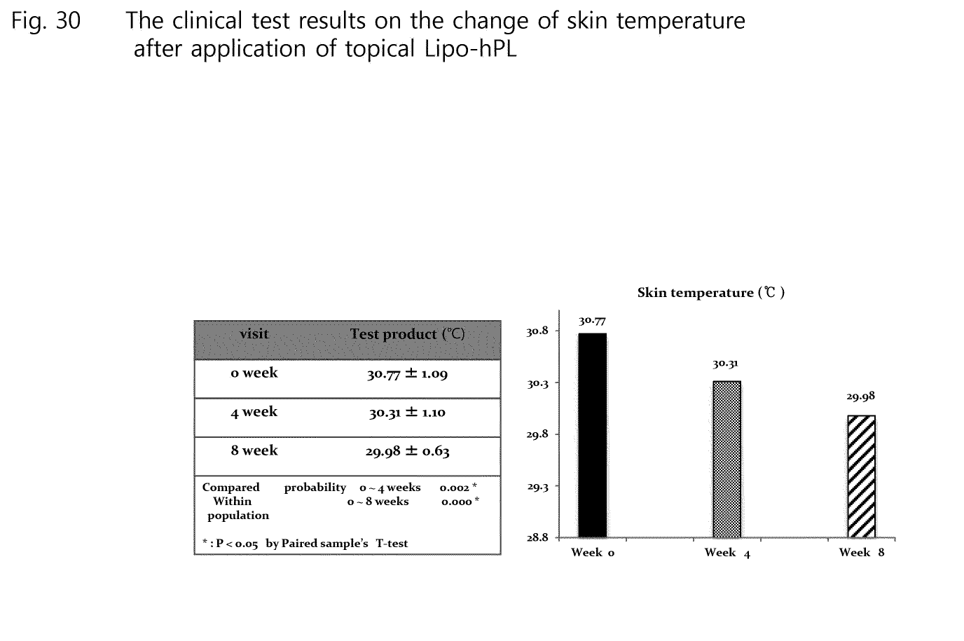

FIG. 31 shows the clinical test results on the change of skin pH after application of topical Lipo-hPL

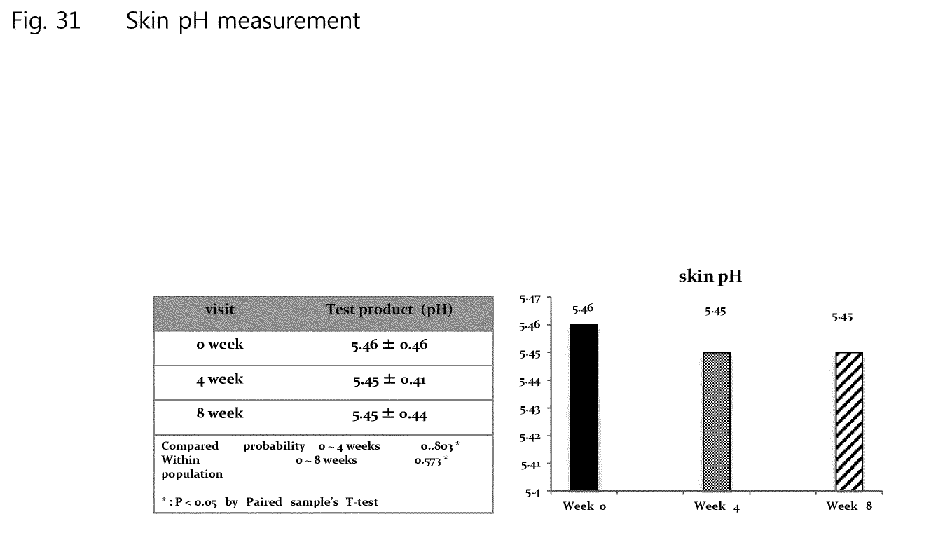

FIG. 32 shows the clinical test results on the evaluation of itchiness after application of topical Lipo-hPL

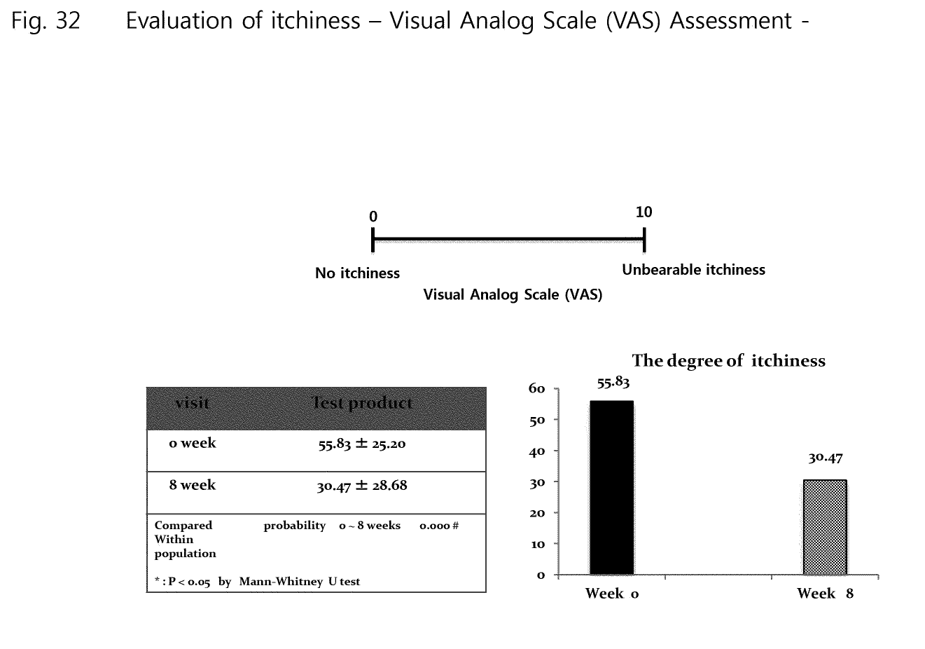

FIG. 33 shows the preference survey test results on the topical Lipo-hPL

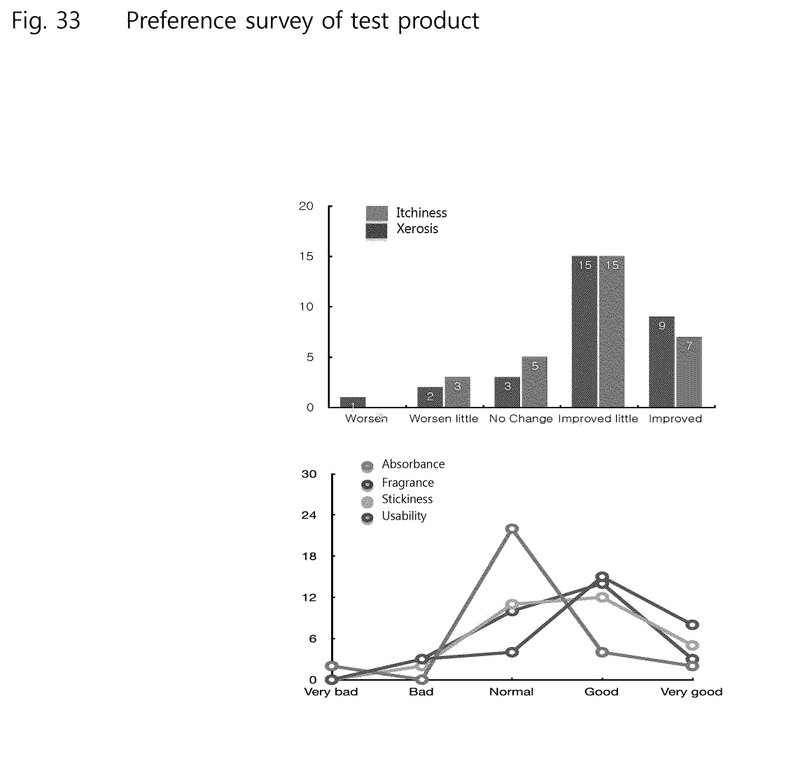

FIG. 34 shows the photographic observation of skin region in atopic-dermatitis patients after application of topical Lipo-hPL

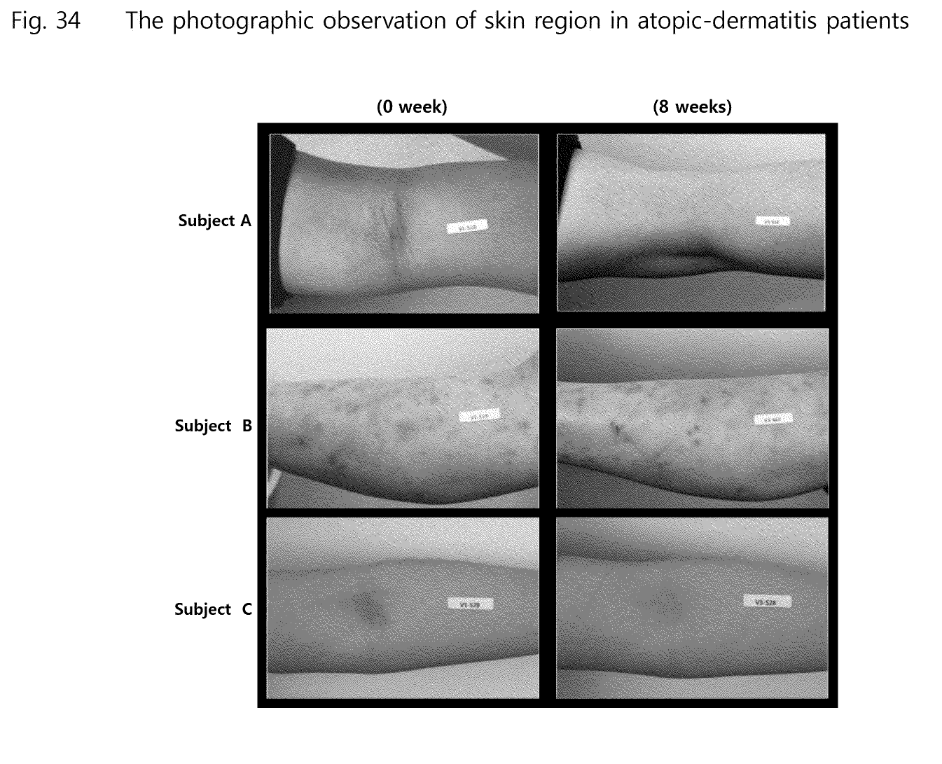

FIG. 35 shows the changes in the skin condition of Nc/Nga mouse with atopic dermatitis after the short term topical application of Lipo-hPL

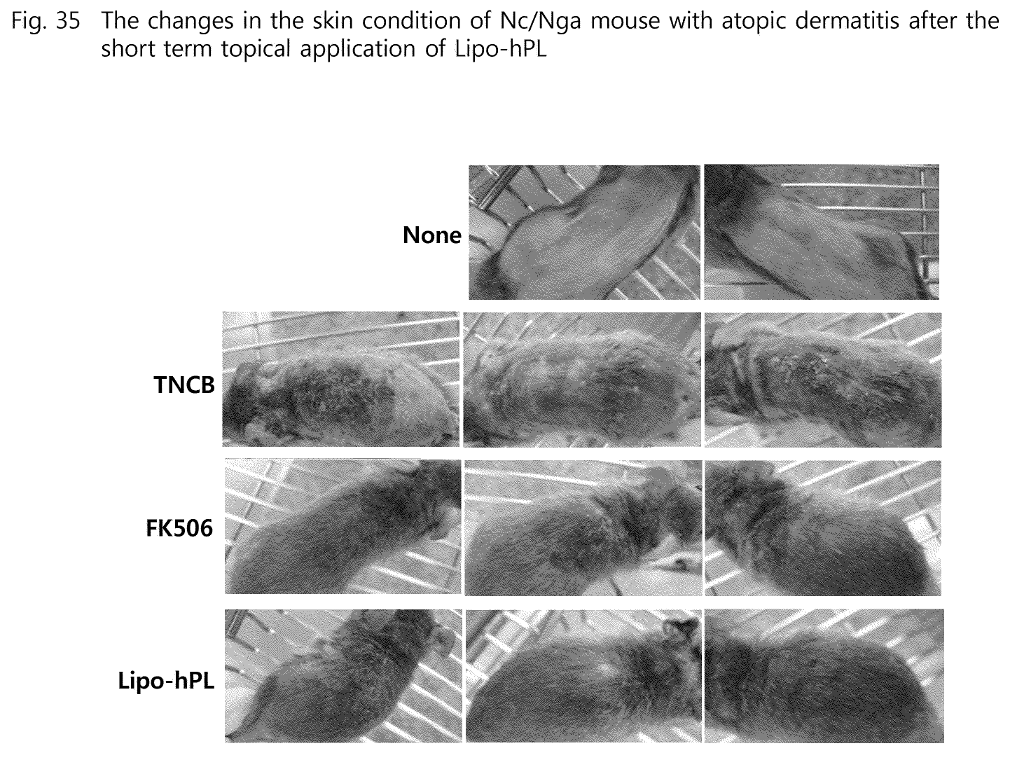

FIG. 36 shows the H&E stain results of changes in the dermal tissue of Nc/Nga mouse with atopic dermatitis after the short term topical application of Lipo-hPL

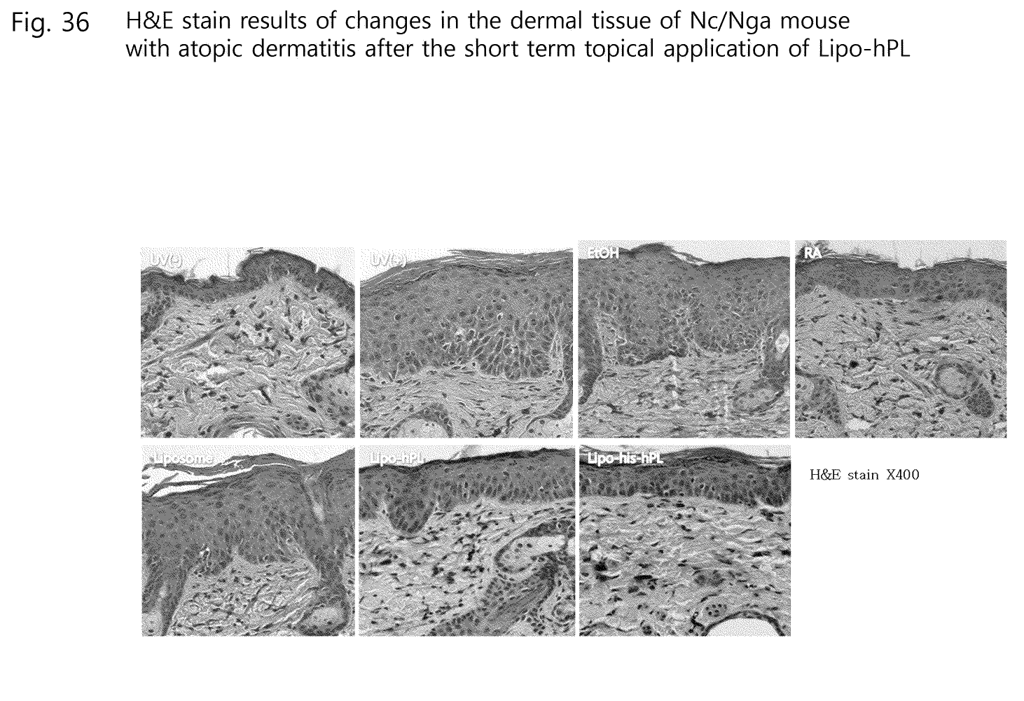

FIG. 37 shows Whitening effect of Lipo-hPL in a cell culture melanin production assay with B16F1 mouse melanoma cell line

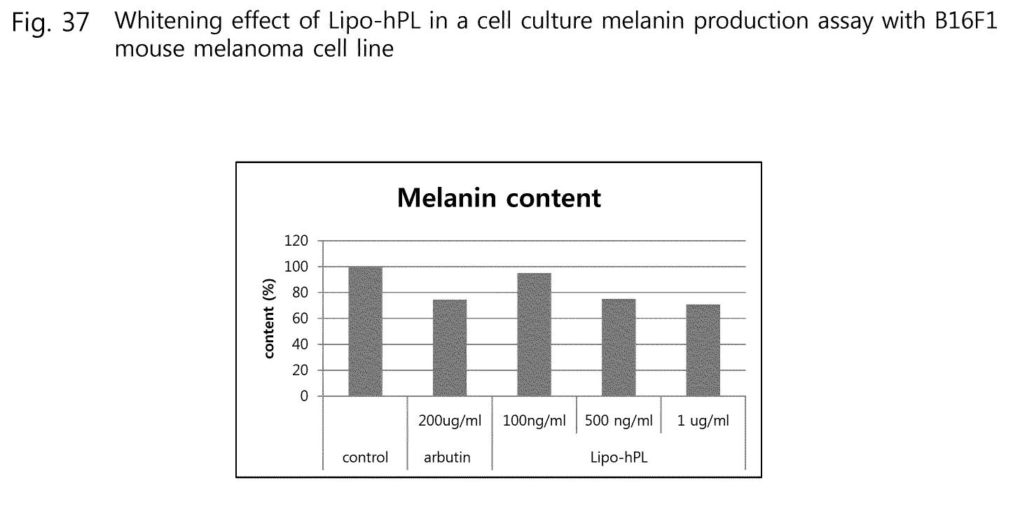

### DETAILED DESCRIPTION OF THE INVENTION

The present invention provides evidence that proteins can be made effective cosmetic active ingredients through their delivery into skin hair follicles by encapsulating them in proper liposome carriers and that beneficial cosmetic as well as pharmaceutical outcomes can be attained through their interaction with their respective receptors on the critical cells including stem cells and/or progenitor cells residing in the skin hair follicles.

The present invention is the first report on the skin-improving composition comprising human placental lactogen as the active ingredient for the treatment and/or improvement of atopic dermatitis, contact dermatitis, skin damages caused by ultraviolet-light exposure, photoaging, UV or sunburn-induced pigmentation, wrinkles, age spots, acne, dryness, itching, actinic keratosis, eczema, psoriasis, sunburn and skin aging; and improving skin elasticity and moisturization by topical application.

Throughout the present invention a number of patent and non-patent references are cited to provide clearer description and explanation of the core techniques and other contents of the claimed arts.

For hPL to have any effect on the skin, the skin cells should have receptors for hPL, as hPL supposedly functions through interaction with its receptors on the cell surface. Given that hPL circulates with the blood in vivo and therefore can interact only with the cells facing the blood, the finding that hPL receptors are practically all over the skin cells making up the hair follicles that normally would not directly contact the blood (i.e., circulating blood), is quite unexpected. Putting available experimental data together, one can come up with a motion picture with the following developments: liposome-encapsulated hPL is applied on the intact skin; hPL-encapsulating liposomes accumulate centering around a hair follicle; hPL-encapsulating liposomes enter the hair follicle, slither down along the hair shaft interacting with the environment on the path inside the hair follicle, subsequently release hPL molecules as the liposomes disintegrate and lose their phospholipid components to the surrounding environment; unshielded hPL molecules interact with hPL receptors expressed on the viable cell surfaces constituting the hair follicular contours; the interacting cells convey signals to themselves and to the adjacent cells that result in observable efficacies, given time; released hPL is exposed to and eventually degraded by abundant proteases present in the hair follicle.

A liposome, especially nano-sized ones, has been suggested to serve as an efficient delivery system for a certain macromolecule into the skin (see, e.g., U.S. Pat. No. 7,951,396). However, considering the compactness of the skin stratum corneum and the size and hydrophilicity of hPL, it is unlikely that a protein macromolecule like hPL, even in a liposome-encapsulated form, exerts its effect on the cells of the skin dermis by directly penetrating through the skin epidermis. A more reasonable view to any effect of hPL attributable to changes in the skin would be as follows: 1) hPL gets delivered into the hair follicle by aid of a liposome carrier and released out of the liposome; 2) the released hPL molecules interact with some of the epidermal basal layer cells making up the inner root sheath of the hair follicle; 3) these interactions cause changes in the interacting cells themselves; 4) in addition, those interactions can directly affect the adjacent cells in direct contact with the interacting cells and also indirectly affect nearby non-touching cells through secretion of (a) secondary mediator(s); 5) these cascading modes of direct and indirect signaling can propagate eventually to the dermal cells without the trigger hPL ever penetrating the epidermal layer or reaching the dermal layer. One important advantage hPL has over small chemicals as a cosmetic ingredient stands out as an outcome of this scenario of events affecting skin conditions; that is, the safest nature of hPL as cosmetic ingredients.

According to the proposed sequence of events, hPL interacts only temporarily with the outermost cells lining the hair follicle, then is going to be degraded completely by the interacting cell itself or by proteases present in the hair follicle. Moreover, the cells that experienced direct encounters with hPL are most likely to undergo natural path to differentiation into keratinocytes that are destined to be peeled off from the skin several weeks later, leaving no adverse or cumulative effects, if any, to the rest of the surviving skin. Except the cells in direct contact with extraneously supplied hPL, all other cells affected by the interaction are experiencing physiological signals that are intrinsic to our body. Even artificially supplied cosmetic hPL is not that foreign to the body. In real sense, therefore, hPL is a truly natural cosmetic ingredient in the point of view of our human skin. And that lack of side effects is exactly what has been observed and concluded with the use of cosmetic hPL of this invention.

Unless otherwise defined, scientific and technical terms used herein related to the present invention shall have the meanings that are commonly understood by those with ordinary skill in the art. Furthermore, unless otherwise required by context, singular terms shall include pluralities and plural terms shall include the singular ones.

The term “effective amount” or correct dosage as used herein means an amount sufficient to achieve the improvement effects in the skin conditions described above.

In accordance with the present invention, there is provided a composition for improving skin conditions, which comprises the effective amount of human placental lactogen (hPL) as the active ingredient. In another aspect of this invention, the composition for the skin condition improvement is encapsulated in a liposome, preferably a nanoliposome. The nanoliposome provided in the present invention is physiologically safe and effective in maintaining the activity of hPL until it reaches the targeted epidermal layers in the hair follicle to promote effective treatments of skin conditions. The present invention further encompasses the methods of improving the skin conditions, such as atopic dermatitis, contact dermatitis, skin damages caused by ultraviolet-light exposure, photoaging, UV or sunburn-induced pigmentation, wrinkles, age spots, acne, dryness, itching, actinic keratosis, eczema, psoriasis, sunburn, skin elasticity problems, and skin aging; and improving skin elasticity and moisturization by topical application of effective amount of the composition with human placental lactogen as an active ingredient. In certain embodiments of the methods of the invention, hair growth and/or hair loss are treated. The present invention further encompasses a large scale production of the recombinant hPL polypeptide useful for formulation and commercialization of hPL-containing compositions effective for improving skin conditions.

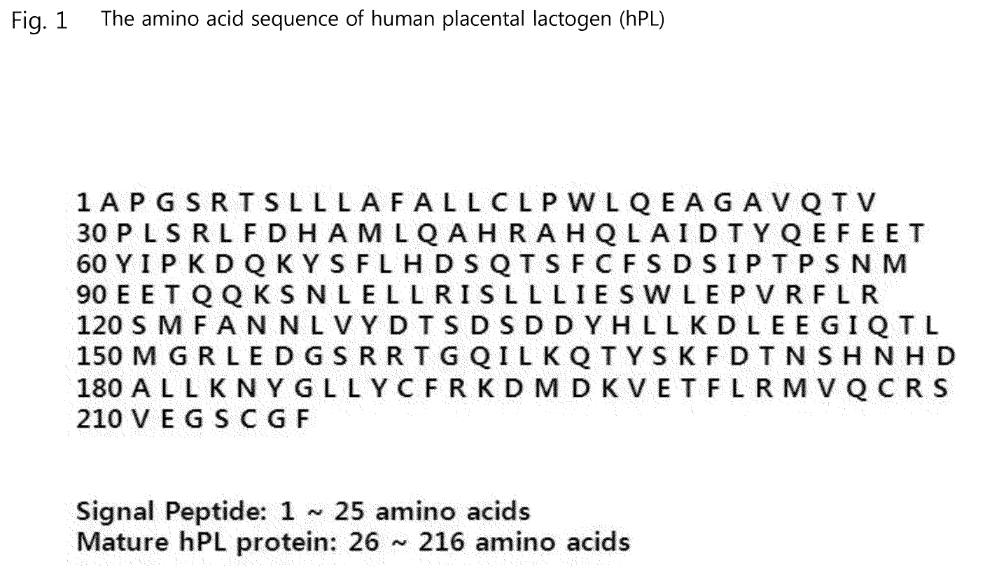

Human placental lactogen (hPL) used as the active ingredient of the skin-improvement composition in the present invention refers to any polypeptides demonstrating the activity of human placental lactogen, such as mature hPL, Met-hPL, hPL variants, modified hPL, hPL analogues, or hPL fragments. Mature hPL and Met-hPL are the preferred composition to obtain the most effective skin improvements. The mature hPL refers to the major hPL encoded by the entire nucleic acid sequence of endogenous human placental lactogen represented in the FIG. 1, whereas Met-hPL has an additional methionin residue at the N-terminal end of the mature hPL. The hPL variants refer to any hPLs having sequences that are different from endogenous hPL. Modified hPLs refer to the hPLs having pegylation (modified with polyethylene glycols), glycosylation or glycation at one or more amino acid residues. The hPL fragments include all hPLs fragments produced by intentionally deleting some part(s) of endogenous hPL through the recombinant DNA technology or artificially synthesized peptides fragments consisting of more than 10 amino acid residues in the sequence that are 100% identical to certain parts of endogenous hPL. The hPL analogues are produced by recombinant DNA technology, which contain some amino acid residues substituted with other amino acids with similar biochemical properties.

As used herein, the phrase “having human placental lactogen activity” can be specified according to one of the following two methods. In one method, it can be specified according to whether human placental lactogen (hPL) causes signaling by binding to hPL-binding proteins or a hPL receptor, and in another method, it can be specified according to whether biological effects caused by the action of hPL can be proven by biological/biochemical methods.

A composition according to a preferred embodiment of the present invention has a phospholipid or liposome composition, and preferably a liposome composition. It is preferable that human placental lactogen as an active ingredient is encapsulated in liposome and applied to the skin. According to a more preferred embodiment of the present invention, the inventive composition has a nanoliposome composition. As used herein, the term “nanoliposome” refers to a liposome having the form of conventional liposome and a mean particle diameter of 20-1000 nm. According to a preferred embodiment of the present invention, the mean particle diameter of the nanoliposome is 50-500 nm, more preferably 50-350 nm, and most preferably 100-250 nm. The nanoliposome utilized for encapsulating hPL in the present invention has been proven to be safe according to measurement of changes in the blood IgE concentration.

Liposome is defined as a spherical phospholipid vesicle of colloidal particles which can be self-associated or self-assembled and composed of amphiphilic molecules each having a water soluble head (hydrophilic group) and a water insoluble tail (hydrophobic group), which show a structure aligned by spontaneous binding caused by the interaction therebetween. Liposomes are classified, according to the size and lamellarity thereof, into SUV (small unilamellar vesicle), LUV (large unilamellar vesicle) and MLV (multi lamellar vesicle). The liposomes showing various lamellarities as described above have a double membrane structure similar to the cell membrane.

The liposome in the present invention can be prepared using phospholipid, polyol, a surfactant, fatty acid, salt and/or water.

Phospholipid which is a component used in the preparation of the inventive liposome is an amphiphilic lipid, and examples thereof include natural phospholipids (e.g., egg yolk lecithin, soybean lecithin, and sphingomyelin) and synthetic phospholipids (e.g., dipalmitoyl-phosphatidylcholine or hydrogenated lecithin), the lecithin being preferred. More preferably, the lecithin is a natural unsaturated or saturated lecithin extracted from soybean or egg yolk.

Polyols which can be used in the preparation of the inventive liposome are not specifically limited and preferably include propylene glycol, dipropylene glycol, 1,3-butylene glycol, glycerin, methylpropanediol, isoprene glycol, pentylene glycol, erythritol, xylitol and sorbitol.

The surfactant which can be used in the preparation of the inventive liposome may be any surfactant known in the art, and examples thereof include anionic surfactants (e.g., alkyl acylglutamate, alkyl phosphate, alkyl lactate, dialkyl phosphate and trialkyl phosphate), cationic surfactants, amphoteric surfactants and nonionic surfactants (e.g., alkoxylated alkylether, alkoxylated alkylester, alkylpolyglycoside, polyglycerylester and sugar ester).

The fatty acids which can be used in the preparation of the inventive liposome are higher fatty acids, and preferably saturated or unsaturated fatty acid having a C₁₂₋₂₂ alkyl chain, and examples thereof include lauric acid, myristic acid, palmitic acid, stearic acid, oleic acid and linoleic acid.

The salt which is used in the preparation of the inventive liposome may be any salt known in the art, regardless of inorganic or organic, and examples thereof include phosphate salt, sulfate salt, nitrate salt, chloride salt, hydroxide salt, sodium salt, potassium salt, calcium salt, ammonium salt, acetate salt, citrate salt, amino acid salt, and amino acid.

Water used in the preparation of the inventive liposome is generally deionized distilled water.

According to a preferred embodiment of the present invention, the inventive liposome is prepared only with phospholipid, salt and water, as described in detail in Examples below.

According to a preferred embodiment of the present invention, the inventive hPL-containing nanoliposome is prepared through a process comprising the steps of: (a) dissolving a phospholipid capable of forming liposome (preferably, egg yolk lecithin or soybean lecithin) in a buffered aqueous solution of salt containing human placental lactogen; and (b) passing the aqueous solution containing human placental lactogen and phospholipid through a high-pressure homogenizer while gradually increasing the content of the phospholipid and the pressure of the high-pressure homogenizer as the number of the passages increases, thus preparing a human placental lactogen-containing nanoliposome (see, e.g., U.S. Pat. No. 7,951,396 and U.S. Patent Publication No. 2008/0213346).

The aqueous solution containing human placental lactogen is preferably a buffer solution having a pH of 6-8, and more preferably about 7, for example, sodium phosphate buffer solution. If the sodium phosphate buffer solution is used, the concentration thereof will preferably be 5-100 mM, more preferably 5-60 mM, even more preferably 10-30 mM, and most preferably about 20 mM.

The most special aspect of the inventive process is that the mixture of the phospholipid and the hPL-containing aqueous solution is passed through the high-pressure homogenizer several times, in which the amount of the phospholipid and the pressure of the homogenizer are gradually increased as the number of the passages increases. According to a preferred embodiment of the present invention, the pressure of the homogenizer is gradually to 0-1000 bar, and more preferably 0-800 bar. The pressure can be increased by 50 bar or 100 bar, and preferably 100 bar. According to a preferred embodiment of the present invention, the amount of the phospholipid is gradually increased to 5-40 w/v (%), and more preferably 5-30 w/v (%).

Through the high-pressure homogenization process including these gradual increases in phospholipid content and pressure, an hPL-containing nanoliposome is prepared and a liquid hPL-containing nanoliposome is preferably prepared (see, e.g., U.S. Pat. No. 5,010,011).

The composition of the present invention is useful in the improvement in various skin conditions. Preferably, the present composition is effective in the improvement in skin conditions including atopic dermatitis, contact dermatitis, skin damages caused by ultraviolet-light exposure, photoaging, UV or sunburn-induced pigmentation, wrinkles, age spots, acne, dryness, itching, actinic keratosis, eczema, psoriasis, sunburn, skin elasticity problems, and skin aging. More specifically, the improvements in skin conditions refer to the treatment of acne and atopic dermatitis, improvement of wrinkles, removal of dark spots, improvement of skin elasticity, prevention of skin aging, improvement of skin moisture and/or moisture-retaining property of skin, suppression of skin irritation or promotion of dermal stem cell proliferation, and the like. More preferably, the skin conditions improved by the present invention include atopic dermatitis, wrinkles and skin damages caused by UV-light.

The present composition may be provided as a cosmetic or pharmaceutical composition.

The present composition includes the hPL encapsulated in nanoliposome (Lipo-hPL) used as the active ingredient, and other components, such as stabilizer, solubilizer, vitamins, dye and fragrance, which are commonly used as auxiliary formulation components of cosmetics.

The cosmetic compositions of the present invention for improving skin conditions may be formulated in a wide variety of forms known in the cosmetic industry, for example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray but not limited to these formulation types. More specifically, the present composition can be formulated to be skin toner, rejuvenating lotion or cream, massaging cream, essence, eye cream, cleansing cream, cleansing foam, cleansing water, mask pack, spray or cosmetic powder. Tables A11-A15 (see the attached Appendix A of Tables A1-A15) provide exemplary pharmaceutical formulations: Table A11 (shampoo), Table A12 (emulsion (lotion)), Table A13 (liquid (toner)), Table A14 (cream)), Table A15 (essence (gel)), and methods for preparing the formulations.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these substances.

In the formulation of powder or spray, it may comprise lactose, talc, silica, aluminum hydroxide, calcium silicate, polyamide powder and mixtures of these substances. Spray may additionally comprise the customary propellants, for example, chlorofluorohydrocarbons, propane/butane or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butylglycol, oils, glycerol fatty esters, polyethylene glycol and fatty acid esters of sorbitan or mixtures of these substances.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these substances.

The formulation of the surfactant containing cleansing products may comprise long chain (fatty) alcohol sulfates, long chain (fatty) alcohol ether sulfates, succinic monoester sulfate, acethionate, imidazolium derivatives, methyltaurate, sarcosinate, fatty acid amide ether sulfates, alkylamido bethane, long chain (fatty) alcohols, fatty acid glycerides, fatty acid diethanolamide, plant seed oils, lanolin derivatives or etoxylated glycerol fatty acid esters.

Where the present composition is formulated to provide a pharmaceutical composition, it may comprise a pharmaceutically acceptable carrier including carbohydrates (e.g., lactose, amylose, dextrose, sucrose, sorbitol, mannitol, starch, cellulose), gum acacia, calcium phosphate, alginate, gelatin, calcium silicate, microcrystalline cellulose, polyvinylpyrrolidone, water, salt solutions, alcohols, gum arabic, syrup, vegetable oils (e.g., corn oil, cotton-seed oil, peanut oil, olive oil, coconut oil), polyethylene glycols, methyl cellulose, methylhydroxy benzoate, propylhydroxy benzoate, talc, magnesium stearate and mineral oil, but not limited to. The pharmaceutical compositions of this invention, further may contain wetting agent, sweetening agent, emulsifier, buffer, suspending agent, preservatives, flavors, perfumes, lubricant, stabilizer, or mixtures of these substances. Details of suitable pharmaceutically acceptable carriers and formulations can be found in Remington's The Science and Practice of Pharmacy (21st ed., 2006), which is incorporated herein by reference.

The pharmaceutical composition of this invention is developed for topical application onto skin.

The correct dosage of cosmetic or pharmaceutical compositions of this invention shall be varied according to the particular formulation, the mode of application, age, body weight and sex of the individual, diet, time and duration of administration, physiological condition of the individual, drug combinations, reaction sensitivities, the individual's ability to metabolize drug(s) and severity of the skin (disease) condition. According to a preferred embodiment of this invention, the suitable and effective hPL dosage unit is to topically administer once a day with 0.001-2000 ng/cm² (unit surface area of skin), preferably, 0.01-200 ng/cm², and most preferably, 0.1-20 ng/cm².

According to the conventional techniques known to those skilled in the art, the cosmetic or pharmaceutical compositions of this invention can be formulated with any cosmetically or pharmaceutically acceptable carrier and/or vehicle, respectively, as described above, finally providing several forms including a unit dosage form. Most preferably, the cosmetic or pharmaceutical composition is a topically applicable solution comprising nanoliposomes.

### EXAMPLES

### Example 1

### Preparation of the Human Placental Lactogen (hPL) Protein

1. Preparation of the Exon DNA of hPL

First, the polymerase chain reaction (PCR) was performed to clone the c-DNA of hPL using the genomic hPL DNA as the template, which was obtained from HEK293T (human embryonic kidney) cell line (ATCC CRL-11268). The primers used for PCR were designed in order to generate exon fragments with the sequences of both termini of each fragment can be base-paired with the other fragments, hence to obtain the entire c-DNA sequence of hPL in the proper order, and subsequently to produce the hPL protein. The DNA polymerase used in the cloning was obtained from Stratagene, U.S.A. (Cat. No. 600154-81).

Basically the following PCR methods were used to prepare the exons constituting entire sequence of endogenous hPL c-DNA. The conjugate of exons 1 and 2 with 194 b.p. was obtained by performing PCR using two primers; hPL 1U which includes the sequence of a part of the exon 1, and hPL 2D. All primers used in PCR procedures of this invention were produced by IDT Inc., U.S.A.; the sequences of primers are provided in the Table 1. The PCR was performed for 36 cycles with each cycle for 1 min at 98° C., 1 min at 66° C., 2min. at 72° C. Using the same PCR protocol, the exon 3 of 144 b.p. was prepared with primers hPL 3U and 4D, then exon 4 of 189 b.p. with primers hPL 5U and 6D, and the exon 5 of 219 b.p. with primers hPL 7U and 8D.

The second round of PCR was performed in the same manner described above to prepare the conjugate of exons 1, 2 and 3 by using the hPL 1U and hPL 4D as the primers and the exons 1 and 2 conjugate, and exon 3 as the template which were produced as described above. Using the same PCR protocols, the conjugate of exon 4 and 5 was prepared by using the primers hPL 5U and hPL 8D, and exon 4 and exon 5 as the template.

The third round of PCR was performed in the same protocol to prepare the hPL nucleic acid which includes the signal peptide residues using primers hPL 1U and hPL 8D and the exons 1,2 and 3 conjugate, and the exons 4 and 5 conjugate as the templates produced from the 2^{nd} round of PCR. Subsequently, the nucleic acid encoding the mature hPL protein was prepared by conducting the PCR using all the template residues including the signal peptide residues produced in the third round of PCR and the primers hPL 9U and hPL 8D.

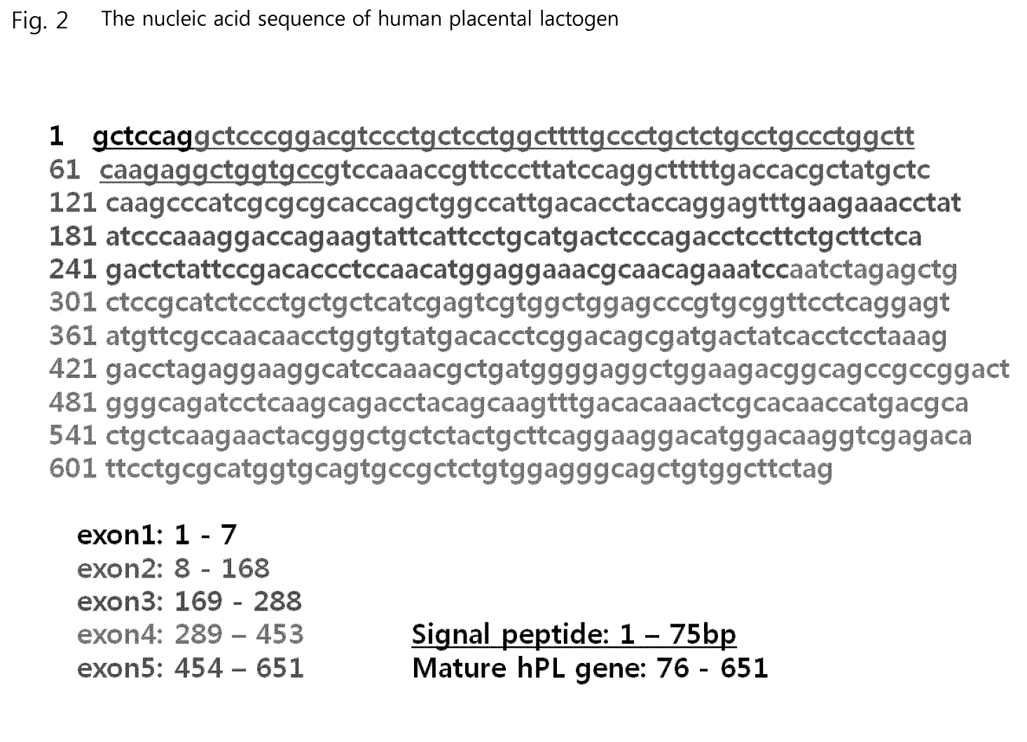

The sequence of mature hPL nucleic acid is shown in the FIG. 2 with each exon highlighted in different colors, and the mature protein sequence in FIG. 1.

The sequences of primers used for PCRs, hPL 1U, hPL 2D, hPL 3U, hPL 4D, hPL 5U, hPL 6D, hPL 7U, hPL 8D and hPL 9U are listed in the Table 1.

TABLE 1
Sequences of primers:

| Primers | Sequences (5′-3′) |
|---|---|
| hPL 1U | gggaattccatatggctccaggctcaaaccgttccc |
| hPL 2D | ataggtttcttcaaactcctggtaggtgtcaatg |
| hPL 3U | taccaggagtttgaagaaacctatatcccaaagg |
| hPL 4D | cagctctagattggatttctgttgcgtttcctc |
| hPL 5U | caacagaaatccaatctagagctgctccgcatc |
| hPL 6D | gtcttccagcctccccatcagcgtttggatg |
| hPL 7U | acgctgatggggaggctggaagacggcagc |
| hPL 8D | cggggtaccctagaagccacagctgccc |
| hPL 9U | gggaattccatatggtccaaaccgttcccttatc |

2. Construction of Expression Vector Containing the Mature hPL Sequence and the narK Promoter

Nucleic acid encoding the Met-hPL and the plasmid pNKmut which includes narK promoter with mutated −10 signal residue (see the published patent application of KR2006-0089086 by Regeron Inc. for more information) were cleaved with Ndel [symbol] Kpnl, then ligated with T4 DNA ligase. The resulting plasmid construct was transfected into the host cell line Top10F′. After the transformant was incubated at 37° C. for 16 hours, 8 colonies randomly selected were cultured to obtain the plasmid from the transformants in order to identify the plasmids that contain pUC-narK and Met-hPL by performing the 1% agarose gel electrophoresis.
3. Preparation and Purification of Met-hPL Protein

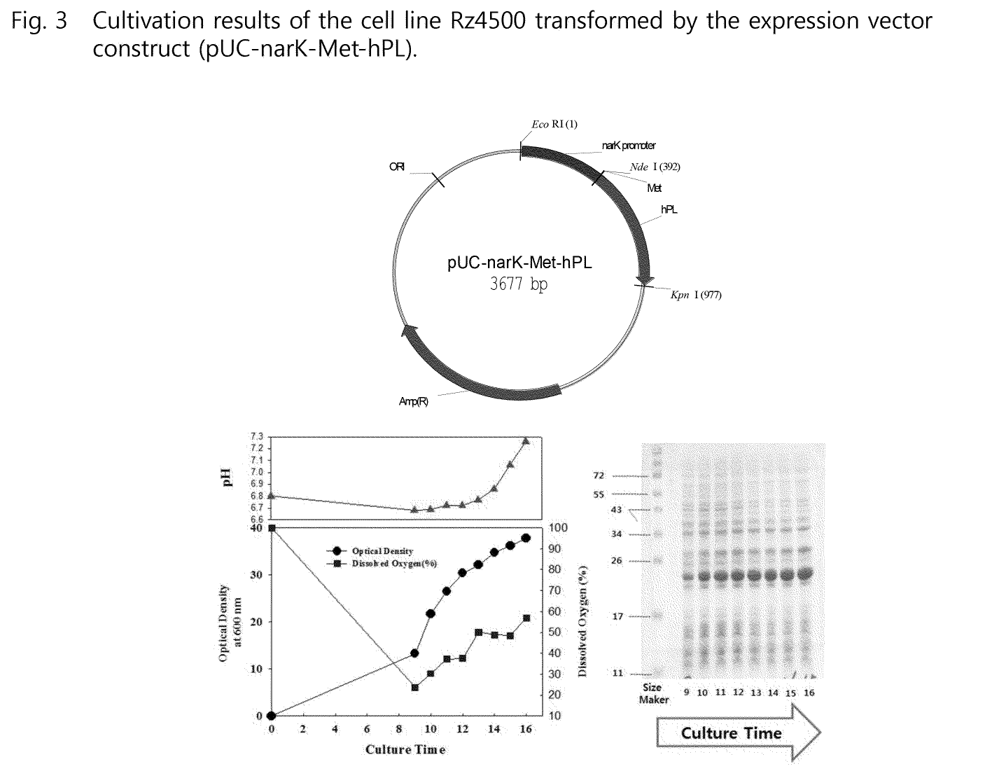

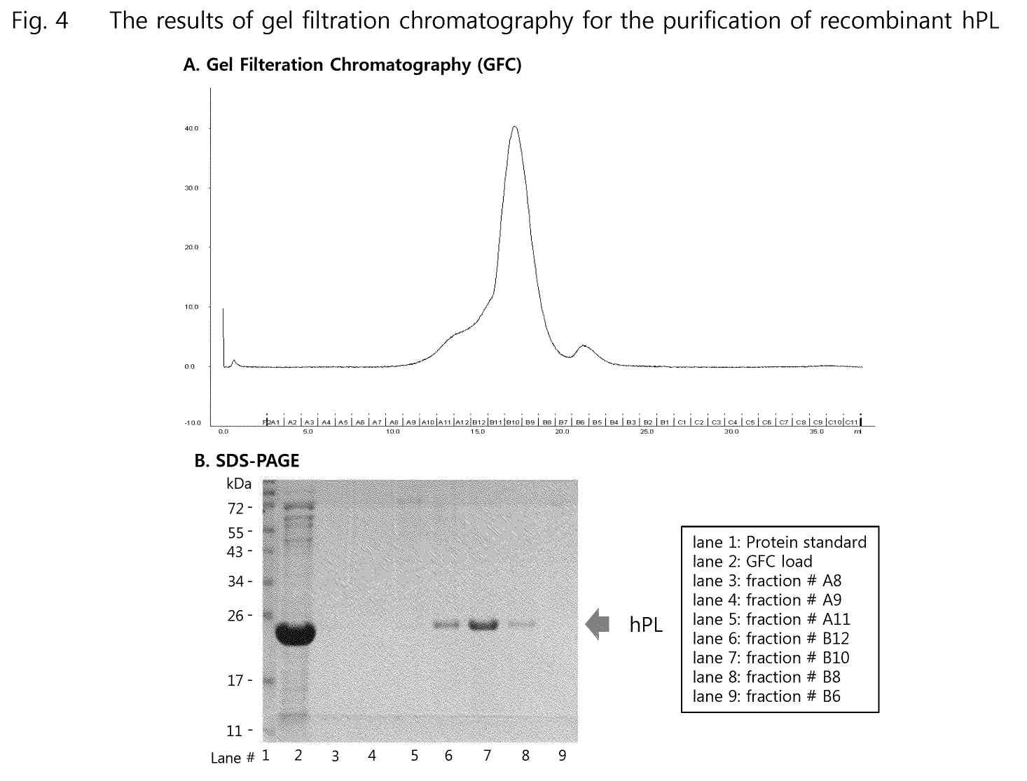

The plasmid construct containing narK promoter and Met-hPL selected from the above procedure was transfected into RZ4500 cell line (provided by Biotechnology Institute of Korea University, Korea), then the transformant was cultured in 7 liter fermentation vessel. FIG. 3 describes the cultivation of the transformed RZ4500 and production of hPL protein as the function of culture time. The cultivated transformant producing the hPL protein was collected by high speed centrifugation, which was subsequently suspended in distilled water followed by ultrasonic lysis. After the lysate was undergone centrifugation, the precipitate was mixed with 0.5% Triton X-100 solution, and recentrifugated. The resulting precipitate is the inclusion body. The inclusion body was mixed with 25 mM NaOH in order to dissolve the proteins, which was neutralized with 10% acetic acid followed by high speed centrifugation to remove impurities. The resulting supernatant was transferred to a gel filtration chromatography column to purify hPL protein. FIG. 4 describes the results of hPL purification using the gel filtration chromatography.

### Example 2

### Preparation of Various Placental Lactogen-Containing Liposome (Lipo-hPL) Formulations

The phospholipids used in the preparation of Lipo-hPL were soybean lecithin (Shindongbang Ltd., Seoul, Korea), Metarin P(Degussa Texturant Systems Deutschland GmbH & Co. KG), Nutripur S(Degussa Texturant Systems Deutschland GmbH & Co. KG), or Emultop (Degussa Texturant Systems Deutschland GmbH & Co. KG).

The heat exchanger of a high-pressure homogenizer (max. output 5 L/hr, highest pressure 1200 bar, Model HS-1002; manufactured by Hwasung Machinery Co., Ltd., South Korea) was placed in ice water such that the temperature of the outlet of the homogenizer did not exceed 30° C., and the inside of the homogenizer was washed with distilled water so as to be ready to operate. Then, to 100 ml of a solution of human placental lactogen dissolved in a buffer solution (20 mM NaH₂PO₄ pH 6.5-7.5, 1 mM EDTA) at a concentration of 1 mg/ml, the phospholipid was added at a ratio of 10 w/v % and sufficiently hydrated and stirred. Then the stirred solution was passed through the homogenizer three times or more at the pressure of 100 bar. To the above homogenized solution, phospholipid was added to a ratio of 18 w/v % and sufficiently hydrated and stirred. The stirred solution was passed through the homogenizer three times or more at 200 bar. Then, to this homogenized solution, phospholipid was added to a ratio of 20 w/v % sufficiently hydrated and stirred, and passed through the homogenizer three times or more at 300 bar. Subsequently, to this solution passed through the homogenizer, phospholipid was added to a ratio of 22 w/v %, sufficiently hydrated and stirred, and passed through the homogenizer three times or more at 400 bar. To the solution passed through the homogenizer in the condition of 400 bar, phospholipid was added to a ratio of 24 w/v %, sufficiently hydrated and stirred, and passed through the homogenizer three times or more at 500 bar. Then, to the solution passed through the homogenizer in the condition of 500 bar, phospholipid was added to a ratio of 26 w/v %, sufficiently hydrated and stirred, and passed through the homogenizer three times or more at 600 bar. Then, to the solution passed through the homogenizer in the condition of 600 bar, phospholipid was added to a ratio of 28 w/v %, sufficiently hydrated and stirred, and passed through the homogenizer three times or more at 700 bar. Then, the solution passed through the homogenizer in the condition of 700 bar was passed through the homogenizer three times or more at 800 bar. Then solution from this final homogenizing step was centrifuged at 15,000×g for 30 minutes, and the supernatant was harvested. Human placental lactogen remain uncapsulated by the liposome after these procedure was separated by gel filtration chromatography to yield the purified Lipo-hPL.

### Example 3

### Preparation of the hPL-Containing Cream Formulation

TABLE 2
The commercial cream formulation containing Lipo-hPL
prepared as described in the Example 2 was produced
by mixing the following ingredients.

| Ingredients | Weight % |
|---|---|
| Lipo-hPL | 2.0 |
| Meadow foam oil | 3.0 |
| Cetearyl alcohol | 1.5 |
| Stearic acid | 1.5 |
| Glycerylstearate | 1.5 |
| Liquid paraffin | 10.0 |
| Bee wax | 2.0 |
| Polysorbate 60 | 0.6 |
| Sorbitan sesquiolate | 2.5 |
| Squalene | 3.0 |
| 1,3 butylene glycol | 3.0 |
| Glycerin | 5.0 |
| Triethanolamine | 0.5 |
| Tocopheryl acetate | 0.5 |
| Preservative, coloring | Appropriate amount |
| Fragrance | Appropriate amount |
| Distilled water | Appropriate amount |
| Total | 100 |

### Example 4

### Evaluation of the Skin Condition Improving Effects for Wrinkles, Skin Elasticity, Moisturizing, and Skin Thickness Using Hairless Nude Mice

1. Experimental Design:

TABLE 3

| Group No. | 1 | 2 | 3 | 4 | 5 | 6 | 7 |
|---|---|---|---|---|---|---|---|
| UV exposure | − | + | + | + | + | + | + |
| Skin- | − | − | EtOH | Retinoic | Liposome | Lipo- | Lipo- |
| conditioning | | | (15%) | acid | 2% | hPL | His-hPL* |
| agents | | | | (RA) | | | |
| Volume of | − | − | 100 | 100 | 300 | 300 | 300 |
| skin- | | | | | | | |
| conditioning | | | | | | | |
| agents (ml) | | | | | | | |

*Lipo-His-hPL represent the hPL containing 6 histidine residues flanking the methionine residue at the N-terminal

2. Preparation of the Animals for In-Vivo Experiments

The 4-week-old SKH-1 female nude mice were purchased from Douyul Biotech, Sungnam, Korea). The animal breeding chambers were kept at 24±0.2° C. and relative humidity at 50±10% in a 12-hr light/12-hr dark cycle. The animals were permitted free access to solid feed (Central Lab. Animal Inc., Seoul, Korea) and water sterilized by irradiation and acclimated for about 2 weeks before divided them into 7 groups by random selection.

In order to induce wrinkle formation on the backside of the nude mice, UVB was irradiated to the mice three times a week for 11 weeks using the VLX-3W stimulator (Vilber Lourmat, Marne la Vallee, France) in the protocol: 30 mJ/cm² for the weeks 1 and 2, 40 mJ/cm² for the weeks 3 and 4, 50 mJ/cm² for the weeks 5 through 9, and 60 mJ/cm² for the weeks 10 and 11. Then, to the UVB-irradiated dorsal area of the animals, 300 μl of the control solution, Lipo-hPL and Lipo-His-hPL were topically applied 5 times a week using a cosmetic brush for 11 weeks, whereas 100 μl EtOH and retinoic acid (RA) were applied for the same frequency. On the day of UVB irradiation, the treatment was performed 1 hour before the irradiation. The concentration of the treating agents is as follows:
3. Concentration of Treating Agents Topically Applied to the Nude Mice:

TABLE 4

| Group | Concentration of treating agents applied | UVB irradiation |
|---|---|---|
| Group 1 | No treatment agent | No |
| Group 2 | No treatment agent | Yes |
| Group 3 | 15% EtOH (100 μl) | Yes |
| Group 4 | 0.1% retinoic acid in 15% EtOH (100 μl) | Yes |
| Group 5 | 2% liposome | Yes |
| Group 6 | 20 μg/ml Lipo-hPL | Yes |
| Group 7 | 4 μg/ml Lipo-his-hPL | Yes |

4. Wrinkle Improving Effects Using Nude Mice

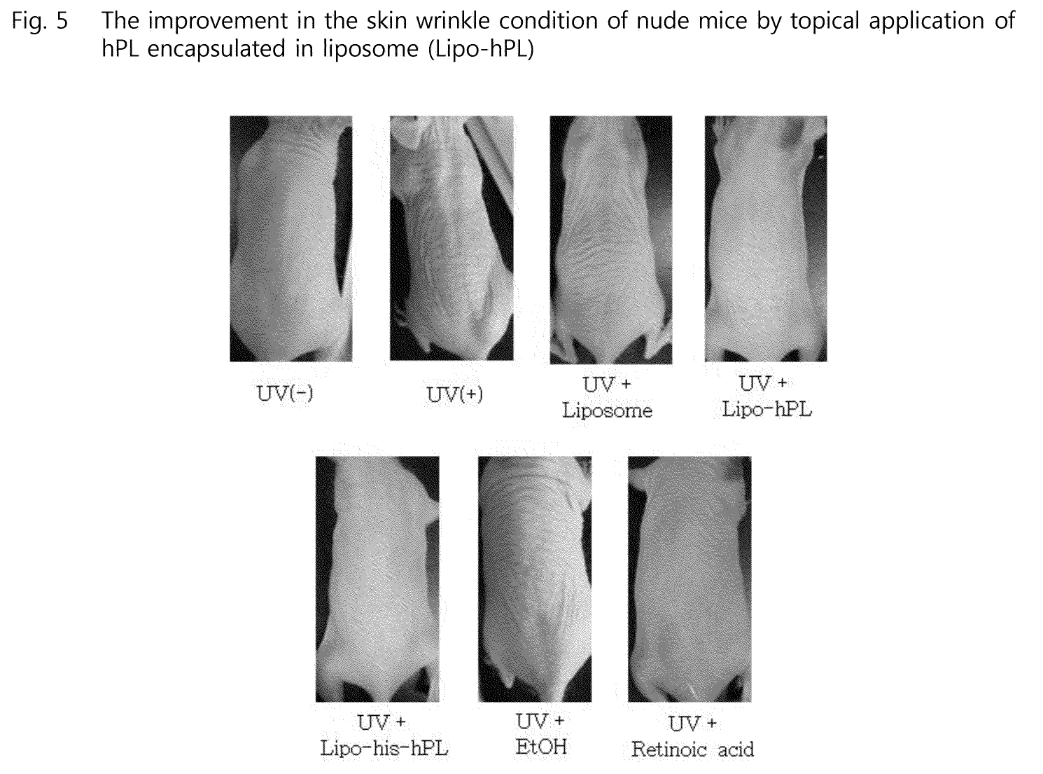

The wrinkle-improving effect was evaluated according to the Donald method (Hyun-Seok Kim et. al, Mech. Ageing Dev. 126:1255-61, 2005). It is well known to those skilled in the art that the prolonged UVB irradiation results in the substantial formation of wrinkles as shown in the SKH-1 hairless nude mice (FIG. 5 UV(+) only). In the present invention, the effects of Lipo-hPL and other skin-conditioning agents on wrinkle formation were evaluated by observing changes of the thickness and depth of wrinkles before and after treatments.

TABLE 5

| Treatment with skin-conditioning | | Changes in wrinkle |
|---|---|---|
| agent | U.V. irradiation | depth and thickness |
| No skin-conditioning agents | No | — |
| No skin-conditioning agents | Yes | ***** |
| Liposome only | Yes | **** |
| Lipo-hPL | Yes | ** |
| Lipo-his-hPL | Yes | ** |
| EtOH only | Yes | ***** |
| Retinoic acid in EtOH | Yes | ** |

Degree of the wrinkle depth and thickness
— No wrinkle formation
* Slightly deep and thick wrinkle
** Slightly to moderately deep and thick wrinkle
*** Moderately deep and thick wrinkle
**** Moderately to severely deep and thick wrinkle
***** Severely deep and thick wrinkle

5. Improvements of Skin Elasticity and Moisturizing Effects by Lipo-hPL

After the completion of 11-weeks UVB irradiation and treatment using various skin-conditioning agents, the effects on the skin elasticity and moisturization of Lipo-hPL were measured in the experimental chamber maintained at 22±2° C. and relative humidity of 50±10%. The mice were acclimated to the environment of the chamber at least 20 minutes prior to the measurement.

Moisturizing effects were analyzed using the Multi Probe Adaptor systems, MPA580 (Courage & Khazaka, Germany) and Texameter, TM300 probe(Courage & Khazaka, Germany) by measuring the loss of moisture in the affected dorsal area. Effects on improving the skin elasticity were evaluated using Cutometer SEM 575 probe (Courage & Khazaka, Germany). All experiments were repeated three times at the settings of Pressure: 500 mbar, Probe aperture: 2 mm, On-time: 1 seconds, Off-time: 1 seconds, Repetitions: 5), then the average of individually measured 3 values was used for evaluating the skin improving effects of Lipo-hPL.

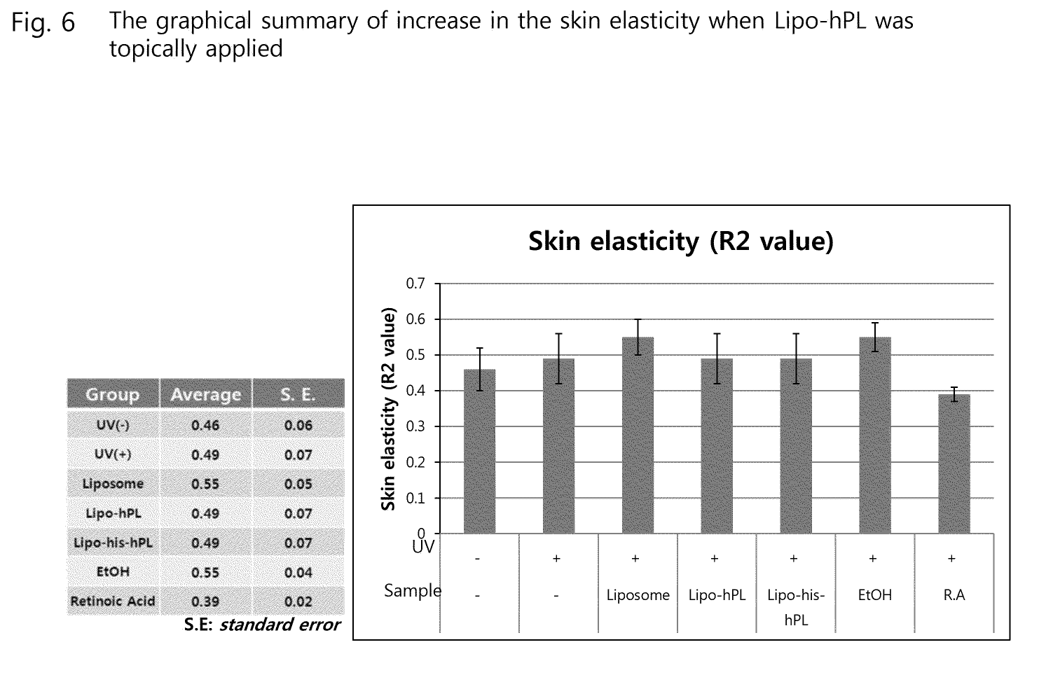

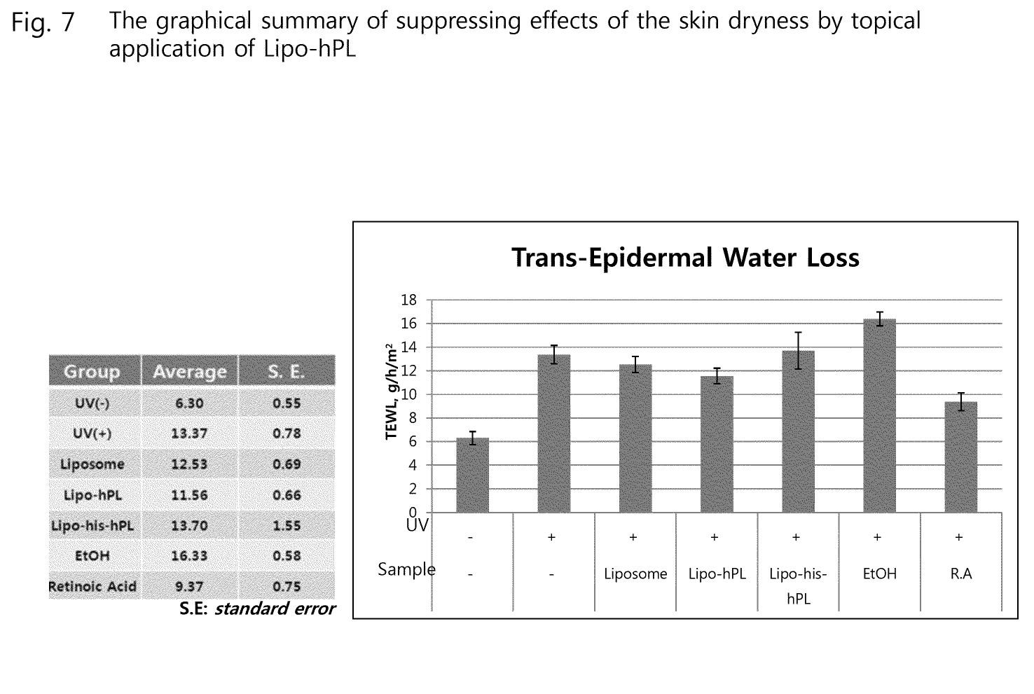

Lipo-hPL and Lipo-his-hPL substantially prevented the loss of moisture when compared to the effects observed in mice treated with EtOH (group 3), UV irradiation only (group 2) or liposome only (group 5) as shown in FIG. 7. Although the effects of improving the skin elasticity per se did not seem to be significant (FIG. 6), the results of other skin improving tests, such as moisturization, reduction of wrinkles, H&E stains, and artificial skin tests suggest that prolonged use of Lipo-hPL (longer than 11 weeks) shall gradually improve the skin elasticity.
6. Artificial Skin Test

Tegoscience Neoderm (Tegoscience., South Korea) was selected as the artificial skin. In routine experiments, 4.5cm² of the artificial skin was treated with 300 μl of 3 different samples for 4 days, each of which contained either one of 1) liposome control (1%), 2) Lipo-his-hPRL (2 ug/ml), or 3) 0.01%. Twenty four hours after the final treatment, the artificial skin was harvested after animals were sacrificed, which was then fixed in 4% formaldehyde for 5 hours at room temperature. After dehydration, the treated tissue was embedded in paraffin (McCormick, U.S.A.) to prepare the paraffin block. The tissue block was then cut into 5 μm thick fragments which were stained with Herris' Hematoxylin (Sigma, U.S.A.) and eosin (Alpha Chem, Inc, U.K.). In addition, it was treated with cytokeratin 14 antibody followed by immunostaining using DAB staining process. Cytokeratin 14 is a differential marker of epithelial cells. Keratinocytes in the basal layer express cytokeratin 14, but this expression is down-regulated during differentiation, switching to cytokeratin 10 as keratinocytes move into suprabasal layers. The results were examined with Zeiss Axiostar Plus microscope before taking the pictures with AxioCam MR camera. The thickness was then measured using the Axiovision software.

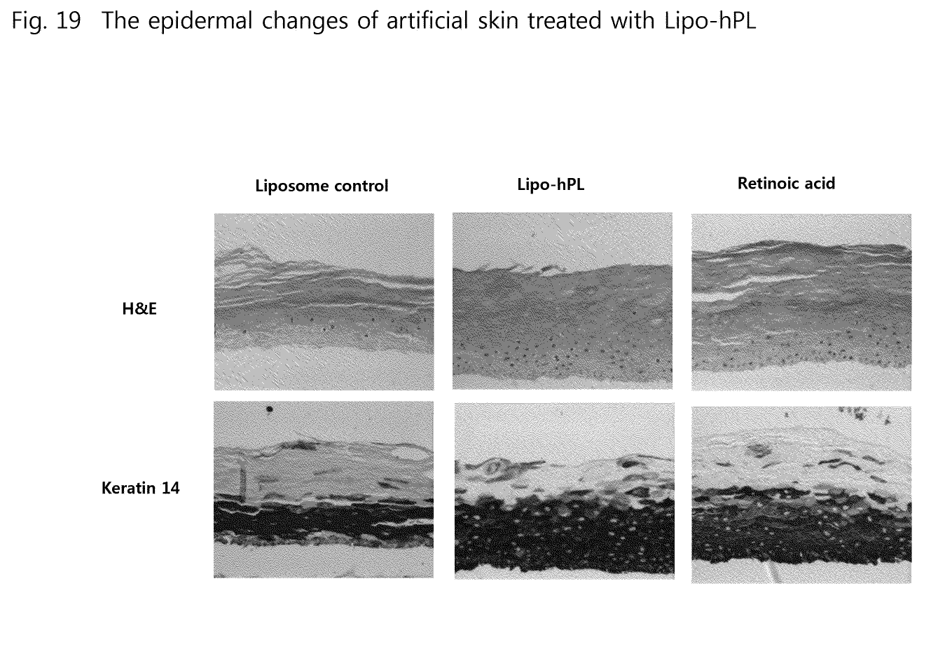

The epidermis of artificial skin treated with Lipo-hPL became significantly thicker. Furthermore, the immunostaining experiment with Cytokeratin 14 confirmed that layers of differentiating cells became thickened upon the treatment with Lipo-hPL. FIG. 19 represents the granular layer, stained in blue, which is located in the middle of epidermis. The one on top of the granular layer is stratum layer. Normally, the thickness of skin layers below stratum layer is considered important in evaluating the skin elasticity.
7. Inhibition of the Skin Damage Due to the UVB Irradiation by Lipo-hPL

Skin thickness of female SKH-1 nude mice was measured by tissue analyses, which were irradiated with UVB and treated with various agents for 11 weeks. The skin tissue of the affected dorsal area was cut and treated overnight by soaking in 0.1 M phosphate fixing solution containing 4% formaldehyde. After dehydration, the treated tissue was fixed in paraffin to prepare a tissue block. The tissue block was then cut into 3 μm thick fragments using Leica microtome, which were subsequently set on glass slides. After soaked in water and deparaffination, the fragments set on slides were stained with Herris' Hematoxylin (Sigma, U.S.A.) and eosin (Alpha Chem, Inc, U.K.) followed by examination with Zeiss Axiostar Plus microscope before taking the pictures with AxioCam MR camera. The thickness was then measured using the Axiovision software.

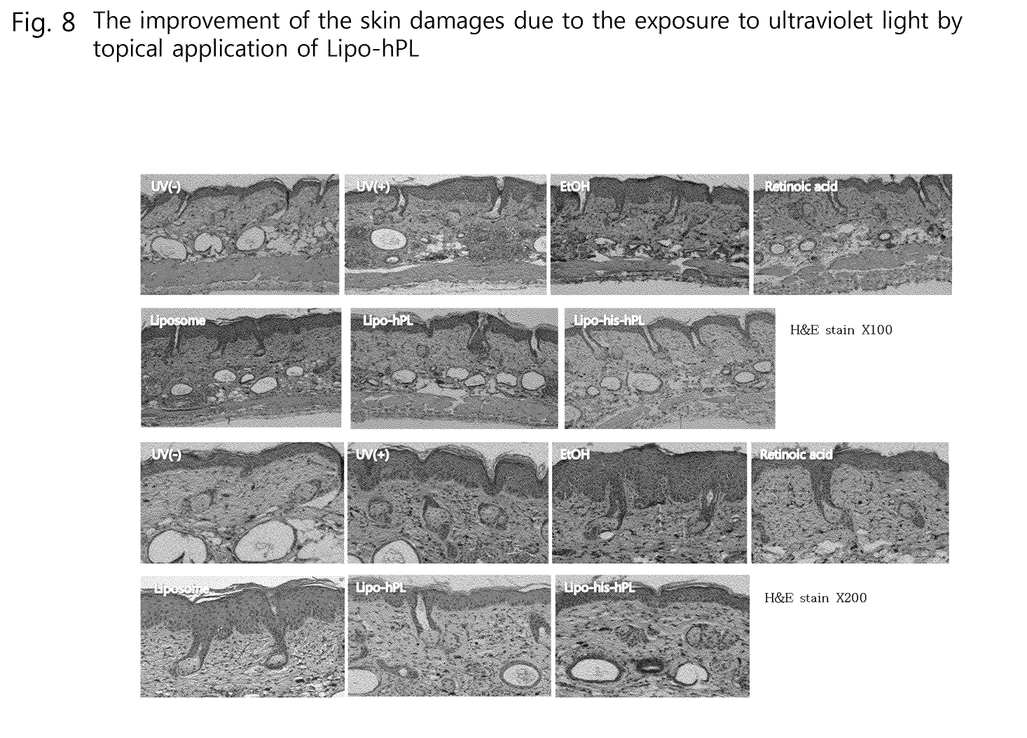

As seen in FIG. 8, not only the wrinkles formation but also the thickening process due to the UVB irradiation were significantly inhibited by the treatment with Lipo-hPL and Lipo-his-hPL. It is suggested that the photo-aging process associated with the abnormal cellular activity during the replication, differentiation and scaling steps might be potentially prevented or delayed by the ability of Lipo-hPL to maintain the normal cellular homeostasis.

### Example 5

### Effects of Lipo-hPL on Healing Atopic Dermatitis

1. Methods of measuring effects of Lipo-hPL on healing skin damages due to short and long term atopic dermatitis
1) Animal Model Used for Skin Inflammation

Four weeks old male Nc/Nga mice were purchased from Orientbio Inc. (Korea) and bred at 24±2° C. and 50±10% relative humidity under 12 hours light/dark cycles. Animals were subject to 1-week acclimating period prior to the experiments.
2) Induction of Inflammation
\textcircled{1} Inflammation Due to the Short Term Atopic Dermatitis

First, the dorsal area of Nc/Nga mice was shaved, which was sensitized by applying 5% TNCB 150 μl. Then 1% TNCB solution made in olive oil was applied to the sensitized area once every week for 6 weeks.
\textcircled{2} Inflammation From the Long Term Atopic Dermatitis

The dorsal area of Nc/Nga mice was shaved, which was sensitized by applying 5% TNCB 150 μl. Then 1% TNCB solution made in olive oil was applied to the sensitized area for total 22 times over the period of 13 weeks.
3) Animal Groups

Animals with short term atopic dermatitis treated with Lipo-hPL: Table 6

TABLE 6

| | Volume of treatment | No. of |
|---|---|---|
| Animal group | agents applied (μl) | animals |
| Group 1: No TNCB treatment, No Lipo-hPL | 0 | 2 |
| Group 2: TNCB treatment; No Lipo-hPL | 150 | 3 |
| Group 3: TNCB treatment; No Lipo-hPL, | 150 | 3 |
| Tacrolimua (FK506) | | |
| Group 4: TNCB treatment; Lipo-hPL | 150 | 3 |
| (20 μg/ml) | | |

Animals with Long Term Atopic Dermatitis Treated with Lipo-hPL: Table 7

TABLE 7

| | Volume of | |
|---|---|---|
| | treatment agents | No. of |
| Animal group | (• l) | animals |
| Group 1: No TNCB treatment, No Lipo-hPL | 0 | 3 |
| Group 2: TNCB treatment; No Lipo-hPL | 150 | 2 |
| Group 3: TNCB treatment; No Lipo-hPL, | 150 | 2 |
| Tacrolimua (FK506) | | |
| Group 4: TNCB treatment; Lipo-hPL (20 μg/ml) | 150 | 2 |

4) Application Methods and Frequencies

Lipo-hPL (20 μg/ml) was topically applied 5 times per week for 6 weeks (short term) and 13 weeks (long term).
5) Methods for Evaluation of the Inflammation and the Efficacy of Lipo-hPL
\textcircled{1} Evaluation of Severity of the Skin Damage

The skin damage was classified to 4 types as described in the Table 8. Based on the pictures taken every week for the inflamed dorsal area, the severity of the damage was evaluated and scored based on the 4 levels as shown below.

The scores and types of skin damage due to inflammation (atopic dermatitis) used for the measurement of the severity of skin damage: Table 8

TABLE 8

| Types of skin damage | Score (degree of inflammation) |
|---|---|
| Erythema | 0 (no symptoms). 1 (mild), 2 (moderate), 3 (severe) |
| Edema | 0 (no symptoms). 1 (mild), 2 (moderate), 3 (severe) |
| Erosion | 0 (no symptoms). 1 (mild), 2 (moderate), 3 (severe) |
| Dryness, scaling | 0 (no symptoms). 1 (mild), 2 (moderate), 3 (severe) |

\textcircled{2} Measurement of Serum IgE

Atopic individuals can have up to 10 times the normal level of IgE in their blood, even though this may not be a requirement for symptoms to occur as has been seen in asthmatics with normal IgE levels in their blood—recent research has shown that IgE production can occur locally in the nasal mucosa (Takhar P et al. (2005) J Immunol 174 (8): 5024-32). After the completion of the Lipo-hPL treatment for the induced inflammation, blood was drawn from the great saphenons vein and the serum was obtained by centrifugation. The serum IgE was measured using the IgE ELISA kit (GomaBiotech, Korea).
\textcircled{3} Evaluation of Changes in the Dermal Tissue

Upon the completion of all necessary treatments, affected areas of the dorsal skin was severed and fixed in 4% formaldehyde and set into a block using paraffin. The paraffin block was cut into 10 um thin slices using microtome, which were set on glass slides followed by stained with hematoxylin and eosin (H&E) before subject to the microscopic observation.
\textcircled{4} Measurement of Skin Thickness

The tissue slides stained with H&E were photographed in order to measure the skin thickness ranging from exoderm, fat layer to the muscle layer using the appropriate software provided for the microscope utilized for the measurement of skin thickness.
2. Results: Efficacy of Lipo-hPL on Healing Skin Damages Due to the Short and Long Term Atopic Dermatitis
1) Evaluation of the Skin Damage
\textcircled{1} Efficacy of Lipo-hPL on Reduction of Skin Damages Due to Short Term Atopic Dermatitis

TABLE 9
Changes in the skin damage symptoms of Nc/Nga mice with short term
atopic dermatitis by the topical application of Lipo-hPL.

| Animal | | | | | Overall | |
|---|---|---|---|---|---|---|
| Treatment | Erythema | Edema | Erosion | Dryness | damage | S.D |
| No TNCB | 0 | 0 | 0 | 0 | 0 | 0 |
| treatment, | | | | | | |
| No Lipo-hPL | | | | | | |
| TNCB treatment; | 8 | 4 | 9 | 6 | 9.0 | 0.8 |
| No Lipo-hPL | | | | | | |
| TNCB treatment; | 3 | 0 | 3 | 3 | 3.0 | 0.3 |
| No Lipo-hPL, | | | | | | |
| but with | | | | | | |
| Tacrolimua | | | | | | |
| (FK506) | | | | | | |
| TNCB treatment; | 3 | 0 | 2 | 3 | 2.7 | 0.2 |
| Lipo-hPL | | | | | | |
| (20 μg/ml) | | | | | | |

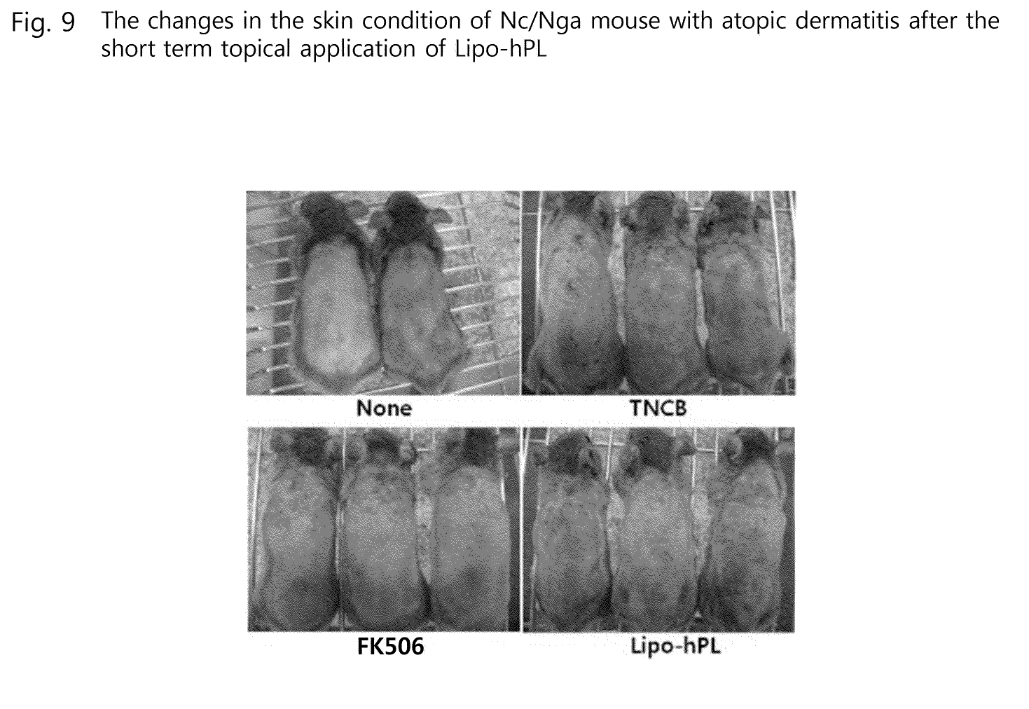

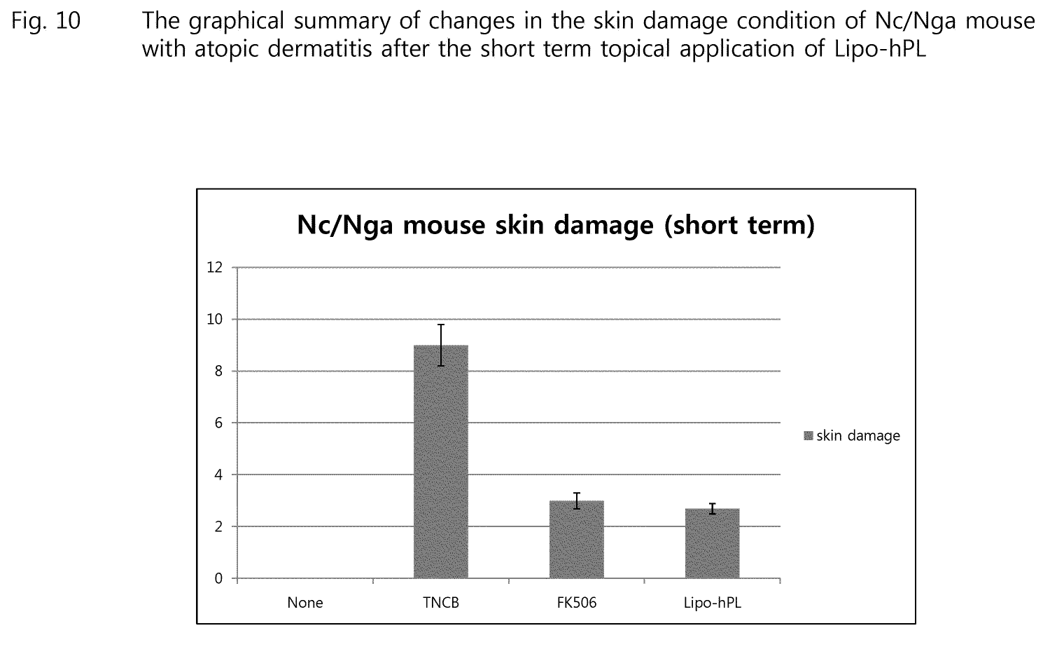

In the Table 9, the values of overall damage (the 5th column) were generated by adding individual skin damage measurements, i.e., Erythema, Edema, Erosion, Dryness shown in the previous columns, then the total of which was divide by the total number of animals (3 in this case) treated. As seen in the Table 9, FIG. 9 and FIG. 10, the topical application of Lipo-hPL significantly reduced skin damages of Nc/Nga mice with short term atopic dermatitis.
\textcircled{2} Efficacy of Lipo-hPL on Reduction of the Skin Damages Due to Long Term Atopic Dermatitis

TABLE 10
Changes in the skin damage symptoms of Nc/Nga mice with long
term atopic dermatitis by the topical application of Lipo-hPL

| Animal | | | | | Overall | |
|---|---|---|---|---|---|---|
| Treatment | Erythema | Edema | Erosion | Dryness | Damage | S.D |
| No TNCB | 0 | 0 | 0 | 0 | 0 | 0 |
| treatment, | | | | | | |
| No Lipo-hPL | | | | | | |
| TNCB treatment; | 2 | 2 | 4.5 | 6 | 7.25 | |
| No Lipo-hPL | | | | | | |
| TNCB treatment; | 1.5 | 1.5 | 3.5 | 3 | 4.75 | |
| No Lipo-hPL, | | | | | | |
| but with | | | | | | |
| Tacrolimua | | | | | | |
| (FK506) | | | | | | |
| TNCB treatment; | 1 | 0 | 0.5 | 1.5 | 1.5 | |
| Lipo-hPL | | | | | | |
| (20 μg/ml) | | | | | | |

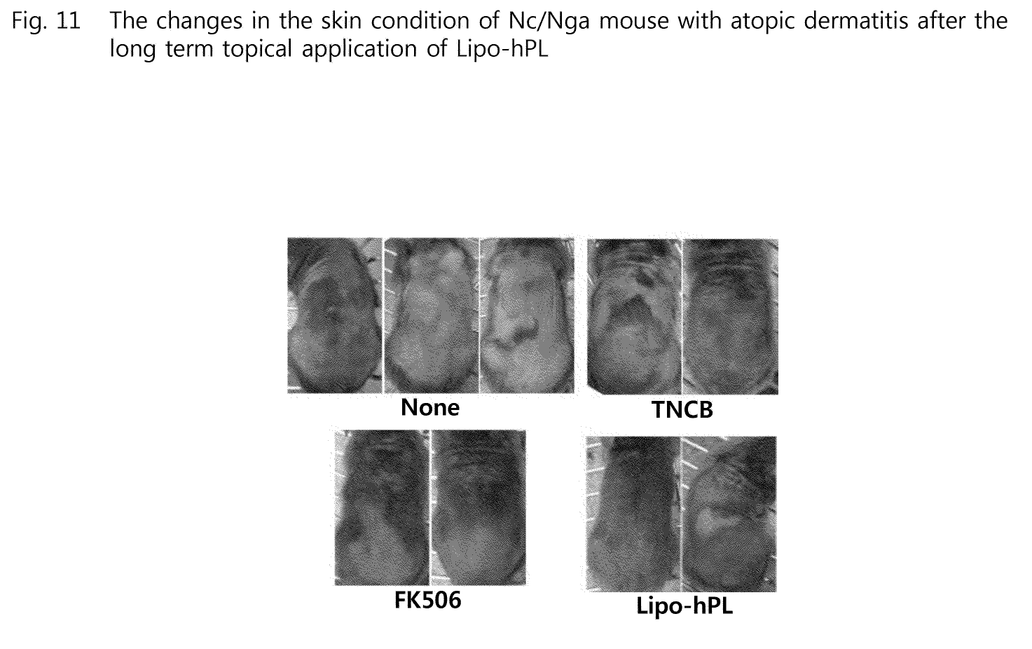

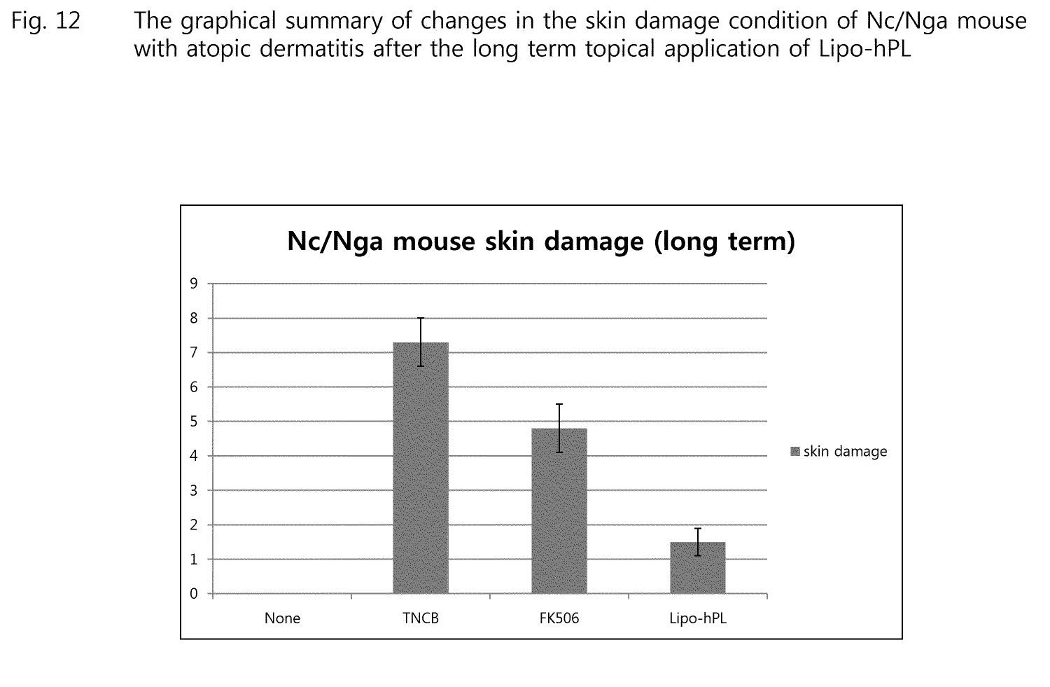

The values of overall damage (the 5th column) were generated by adding individual skin damage measurements, i.e., Erythema, Edema, Erosion, Dryness shown in the previous columns, then the total of which was divide by the total number of animals (2 in this case) treated. As seen in the Table 10, FIG. 11 and FIG. 12, the topical application of Lipo-hPL significantly reduced skin damages of Nc/Nga mice with long term atopic dermatitis.
2) Results of the Serum IgE Measurement

TABLE 11
Results of the serum IgE measurement

| Treatment | N | IgE (ng/ml) | S.D. |
|---|---|---|---|
| No TNCB treatment, No Lipo-hPL | 2 | 3.7 | 0.7 |
| TNCB treatment; No Lipo-hPL | 5 | 99.9 | 16.9 |
| TNCB treatment; No Lipo-hPL, | 3 | 107.1 | 10.6 |
| With Tacrolimua (FK506) | | | |
| TNCB treatment; Lipo-hPL | 3 | 71.0 | 17.6 |
| (20 μg/ml) | | | |

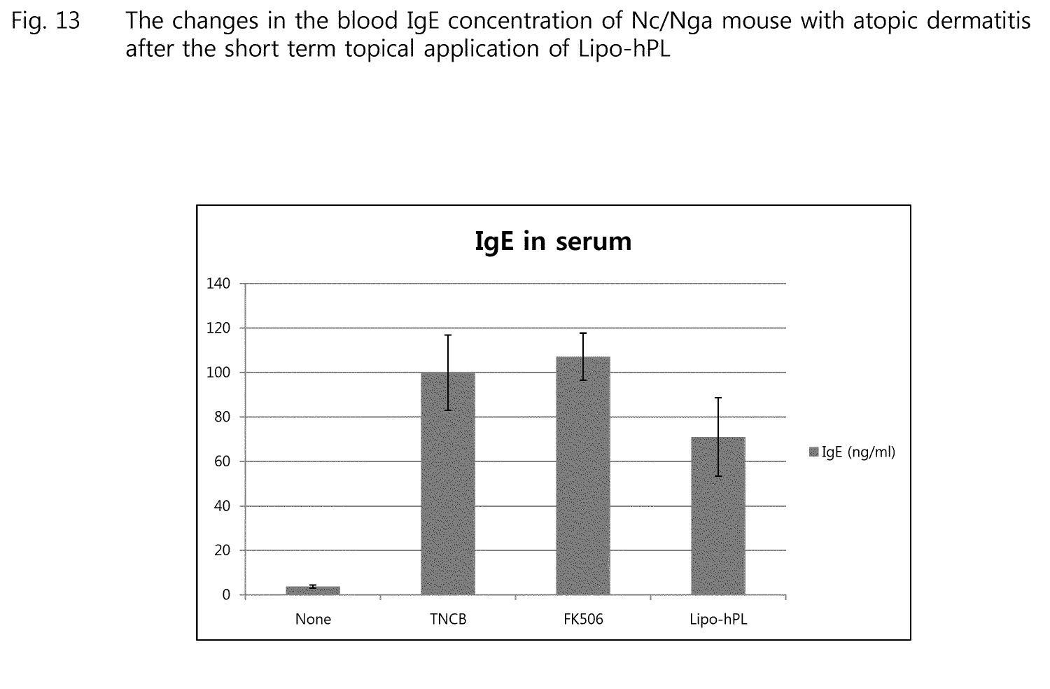

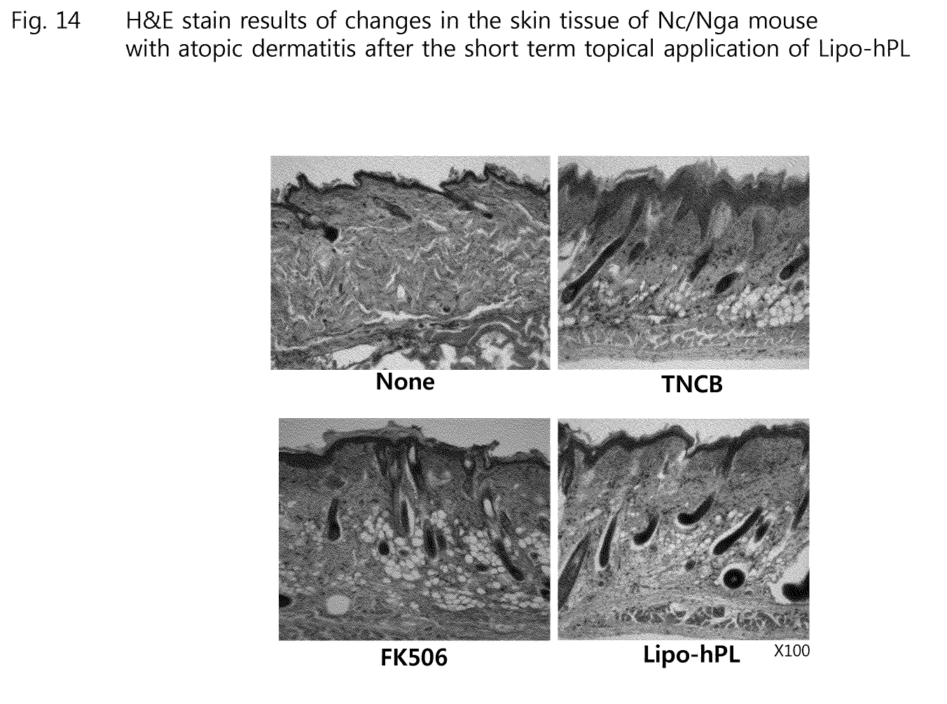

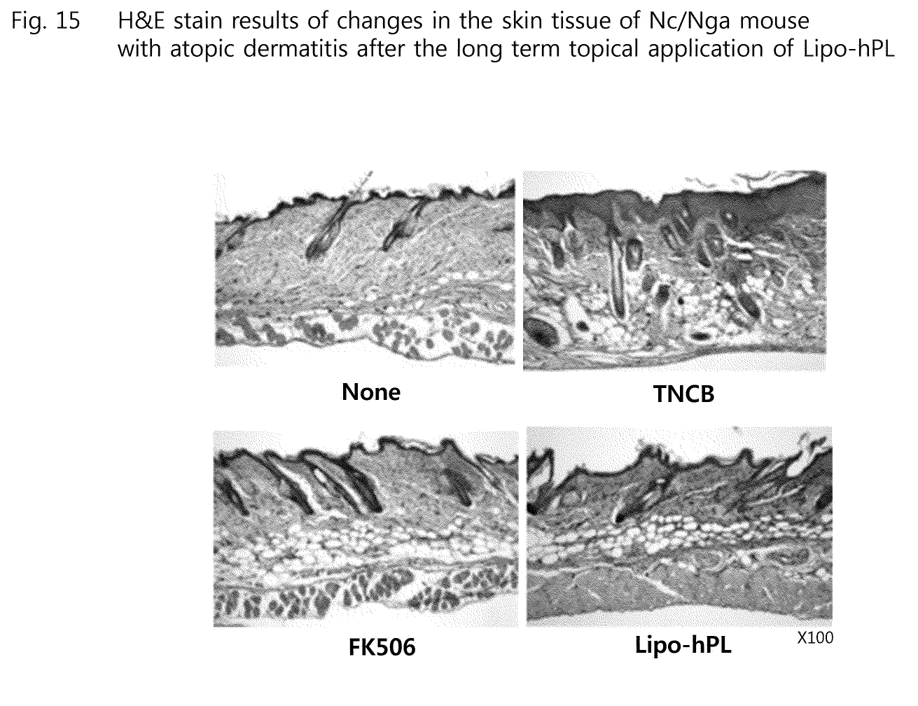

As seen in the Table 11 and FIG. 13, the topical application of Lipo-hPL significantly reduced the serum IgE level when compared to the results for the application of TNCB only or FK506.
3) Changes in the Dermal Tissue
\textcircled{1} Effects of the Short Term Treatment by Lipo-hPL is Shown in FIG. 14
\textcircled{2} Effects of the Long Term Treatment by Lipo-hPL is Shown in FIG. 15

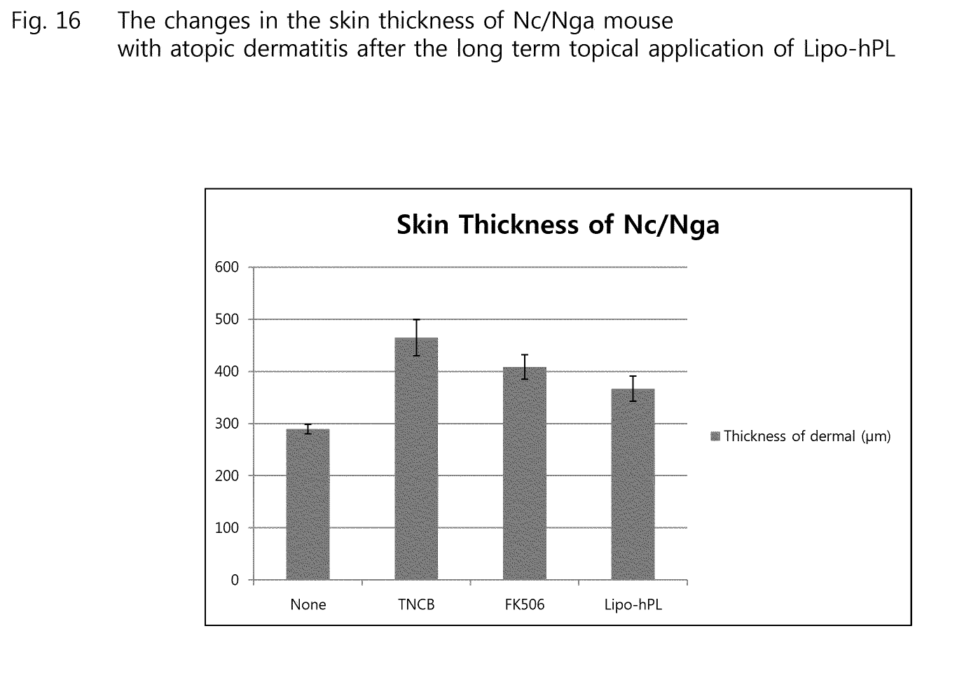

The short and long term treatments indicate the period of the topical application of Lipo-hPL containing 1% TNCB after the pretreatment with 5% TNCB.
4) Changes in the Thickness of Skin with Induced Atopic Dermatitis After the Topical Application of Lipo-hPL is Shown in FIG. 16
3. Results and Discussion of the Efficacy of Lipo-hPL on Skin Damages Due to Atopic Dermatitis
1) Efficacy of the Topical Application of Lipo-hPL on Skin Damages Due to Short and Long Term Atopic Dermatitis
\textcircled{1} Efficacy of Lipo-hPL on Skin Damages Due to Atopic Dermatitis

The values of overall damage (the 5th column) were generated by adding individual skin damages, i.e., Erythema, Edema, Erosion, Dryness shown in the previous columns, then the total of which was divide by the total number of animals (3, in this case) treated.

When compared with the control group, the short term skin damages of Nc/Nga mice due to the application of TNCB only was scored to be 9.0±0.8, out of 10 as the most severe damage. The score was reduced to 3.0±0.3 when FK506 was applied, which was further reduced to 2.7±0.2 upon topical application of Lipo-hPL. On the other hand the long term skin damages of Nc/Nga mice due to the application of TNCB only was scored to be 3.6±0.7, out of 10 as the most severe damage. The score was reduced to 2.4±0.7 when FK506 was applied, which was further reduced to 0.8±0.4 upon topical application of Lipo-hPL. These results suggest that the short and long term topical application of Lipo-hPL is effectively improve skin damages due to atopic dermatitis.
\textcircled{2} Changes in Thickness of Exodermal and Dermal Tissues

The average skin thickness of control animals was 289.5±9.3 μm which was increased by 1.6 folds to 464.9±34.8 pm animals were treated with TNCB to induce the inflammation. Subsequent topical application of FK506 reduced the thickness to 408.0±23.4 pm, which was further reduced to 366.8±24.2 pm by Lipo-hPL (the skin thickness is generated by measuring those of epidermis, dermis, and subcutaneous layer). On the other hand, the exodermal thickness of animals treated with TNCB only was approximately 10 folds thicker than those of the control group, or animals treated with FK506 or Lipo-hPL.
\textcircled{3} Measurement of Serum IgE

Atopic individuals can have up to 10 times the normal level of IgE in their blood, even though this may not be a requirement for symptoms to occur. As seen in the Table 11 and FIG. 13, the control group demonstrated the lowest IgE in ng/ml (3.7±0.7), while the highest serum IgE value was observed for animals treated with TNCB only and followed by those treated with FK506, a steroid type immune suppressant. The serum IgE of TNCB only-treated group was 99.9±16.9 ng/ml, which was reduced to 71.0±17.6 upon treatment with Lipo-hPL.
\textcircled{4} Analysis of Dermal Tissue with H&E Staining

Unlike the control group, TNCB-treated animals demonstrated abnormal cellular behaviors, such as erosion (or infiltration) of immune cells with increased amount of eosinophile and multi-direction movement of red blood cells. The topical application of Lipo-hPL, however, somewhat reduced the amount of eosinophile in dermal tissue. Furthermore, the Lipo-hPL-treated group demonstrated the pattern similar to those of normal control group in development of keratinoid.

### CONCLUSIONS

The topical application of Lipo-hPL significantly ameliorated the skin damages caused by short and long term inflammation due to the exposure to the atopy inducing agent, hence improved skin conditions by suppressing the exodermal hypertrophy and keratinization.

### REFERENCES

| Patent Number (Publication Number) | Filing date |
|---|---|
| U.S. Pat. No. 5,010,011 | Apr. 21, 1988 |
| U.S. Pat. No. 6,136,562 | Nov. 20, 1992 |
| U.S. Pat. No. 7,951,396 | Mar. 28, 2006 |
| US20020032154 | Jun. 7, 2001 |
| US20080213346 | Mar. 28, 2006 |
| US2013035289 | Feb. 16, 2011 |

### Foreign Patent Documents

| Publication Number | Filing date |
|---|---|
| KR20110120254 | Apr. 28, 2011 |

### Other References

Bos J. D. et al., “The 500 Dalton rule for the skin penetration of chemical compounds and drugs”, Experimental Dermatology, 2000, 9(3), 165-169.
Corbacho A M et al., “Roles of prolactin and related members of the prolactin/growth hormone/placental lactogen family in angiogenesis”, J. endocrinol. 2002, 173(2):219-238.
Forsyth I A, “Placental lactogen and prolactin--molecular and functional evolution”, J Mammary Gland Biol Neoplasia. 2002, 7(3):291-312.
Fowlkes J et al., “Placental lactogen-binding sites in isolated fetal fibroblasts: characterization, processing, and regulation”, Endocrinology, 1993, 132 (6):.2477-2483.
Fowlkes J et al., “Binding of placental lactogen and placental lactogen to fetal sheep fibroblasts”, Pediatric research, 1992, 32 (2): 200-203.
Gertler A, “Recombinant analogues of prolactin, growth hormone, and placental lactogen: correlations between physical structure, binding characteristics, and activity”, J Mammary Gland Biol Neoplasia., 1997, 2(1):69-80.
Guyton et al., Textbook of Medical Physiology (11 ed.). Philadelphia: Saunders, 2005 pp. 1033. (ISBN 81-8147-920-3).
Kim H S et al., “Hydroxyurea-induced senescence of human fibroblasts.” Mech. Ageing Dev., 2005, 126:1255-61.
Kossiakoff A A, “The structural basis for biological signaling, regulation, and specificity in the growth hormone-prolactin system of hormones and receptors”. Adv Protein Chem., 2004, 68:147-169.
Lauer A. C. et al., “Targeted delivery to the pilosebaceous unit via liposomes” Advanced drug delivery reviews v.18 no.3, pp.311-324, 1996
Meidan V M et al., “Transfollicular drug delivery is it a reality?, Int J Pharm. 2005, 306(1-2), 1-14
Takhar P et al. “Allergen drives class switching to IgE in the nasal mucosa in allergic rhinitis”. J Immun. (2005) 174 (8): 5024-32.
Troy D. B. (ed)., Remington: The Science and Practice of Pharmacy (21st ed., 2006), Lippincott Williams & Wilkine
Wosickaa H. et al., “Targeting to the hair follicles: Current status and potential” J Dermatological Science 57 (2010) 83-89.

The Tables A1-A15 attached hereto as Appendix A are incorporated herein by reference in their entirety.

Each and every issued patent, patent application and publication referred to herein is hereby incorporated herein by reference in its entirety.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

TABLE A1
Sequences of primers

| Primers | Sequences (5′-3′) |
|---|---|
| hPL 1U | gggaattccatatggctccaggctcaaaccgttccc |
| hPL 2D | ataggtttcttcaaactcctggtaggtgtcaatg |
| hPL 3U | taccaggagtttgaagaaacctatatcccaaagg |
| hPL 4D | cagctctagattggatttctgttgcgtttcctc |
| hPL 5U | caacagaaatccaatctagagctgctccgcatc |
| hPL 6D | gtcttccagcctccccatcagcgtttggatg |
| hPL 7U | acgctgatggggaggctggaagacggcagc |
| hPL 8D | cggggtaccctagaagccacagctgccc |
| hPL 9U | gggaattccatatggtccaaaccgttcccttatc |

APPENDIX A

TABLE A2
The commercial cream formulation containing Lipo-hPL prepared as described
in the Example II was produced by mixing the following ingredients.

| Ingredients | Weight % |
|---|---|
| Lipo-hPL | 2.0 |
| Meadow foam oil | 3.0 |
| Cetearyl alcohol | 1.5 |
| Stearic acid | 1.5 |
| Glycerylstearate | 1.5 |
| Liquid paraffin | 10.0 |
| Bee wax | 2.0 |
| Polysorbate 60 | 0.6 |
| Sorbitan sesquiolate | 2.5 |
| Squalene | 3.0 |
| 1,3-butylene glycol | 3.0 |
| Glycerin | 5.0 |
| Triethanolamine | 0.5 |
| Tocopheryl acetate | 0.5 |
| Preservative, coloring | Appropriate amount |
| Fragrance | Appropriate amount |
| Distilled water | Appropriate amount |
| Total | 100 |

TABLE A3
Hairless nude mouse group for evaluation of the skin
condition improving effects for wrinkles, skin
elasticity, moisturizing, and skin thickness

| Group No. | 1 | 2 | 3 | 4 | 5 | 6 | 7 |
|---|---|---|---|---|---|---|---|
| UV exposure | − | + | + | + | + | + | + |
| Skin- | − | − | EtOH | Retinoic | Liposome | Lipo-hPL | Lipo- |
| conditioning | | | (15%) | acid | 2% | | His- |
| agents | | | | (RA) | | | hPL* |
| Volume of | − | − | 100 | 100 | 300 | 300 | 300 |
| skin- | | | | | | | |
| conditioning | | | | | | | |
| agents (μl) | | | | | | | |

*Lipo-His-hPL represent the hPL containing 6 histidine residues flanking the methionine residue at the N-terminal

TABLE A4
Concentration of treating agents topically applied to the nude mice

| Group | Concentration of treating agents applied | UVB irradiation |
|---|---|---|
| Group 1 | No treatment agent | No |
| Group 2 | No treatment agent | Yes |
| Group 3 | 15% EtOH (100 μl) | Yes |
| Group 4 | 0.1% retinoic acid in 15% EtOH (100 μl) | Yes |
| Group 5 | 2% liposome | Yes |
| Group 6 | 20 μg/ml Lipo-hPL | Yes |
| Group 7 | 4 μg/ml Lipo-his-hPL | Yes |

TABLE A5
Wrinkle improving effects using nude mice

| | U.V. | Changes in wrinkle |
|---|---|---|
| Treatment with skin-conditioning agent | irradiation | depth and thickness |
| No skin-conditioning agents | No | — |
| No skin-conditioning agents | Yes | ***** |
| Liposome only | Yes | **** |
| Lipo-hPL | Yes | ** |
| Lipo-his-hPL | Yes | ** |
| EtOH only | Yes | ***** |
| Retinoic acid in EtOH | Yes | ** |

Degree of the wrinkle depth and thickness
— No wrinkle formation
* Slightly deep and thick wrinkle
** Slightly to moderately deep and thick wrinkle
*** Moderately deep and thick wrinkle
**** Moderately to severely deep and thick wrinkle
***** Severely deep and thick wrinkle

TABLE A6
Animals with short term atopic dermatitis treated with Lipo-hPL

| | Volume of | |
|---|---|---|
| | treatment agents | No. of |
| Animal group | applied (μl) | animals |
| Group 1: No TNCB treatment; No Lipo-hPL | 0 | 2 |
| Group 2: TNCB treatment; No Lipo-hPL | 150 | 3 |
| Group 3: TNCB treatment; No Lipo-hPL, | 150 | 3 |
| Tacrolimua (FK506) | | |
| Group 4: TNCB treatment; Lipo-hPL | 150 | 3 |
| (20 μg/ml) | | |

TABLE A7
Animals with long term atopic dermatitis treated with Lipo-hPL

| | Volume of | |
|---|---|---|
| | treatment agents | No. of |
| Animal group | applied (μl) | animals |
| Group 1: No TNCB treatment; No Lipo-hPL | 0 | 3 |
| Group 2: TNCB treatment; No Lipo-hPL | 150 | 2 |
| Group 3: TNCB treatment; No Lipo-hPL, | 150 | 2 |
| Tacrolimua (FK506) | | |
| Group 4: TNCB treatment; Lipo-hPL | 150 | 2 |
| (20 μg/ml) | | |

TABLE A8
The scores and types of skin damage due to inflammation (atopic dermatitis)
used for the measurement of the severity of skin damage

| Types of skin damage | Score (degree of inflammation) |
|---|---|
| Erythema | 0 (no symptoms). 1 (mild), 2 (moderate), 3 (severe) |
| Edema | 0 (no symptoms). 1 (mild), 2 (moderate), 3 (severe) |
| Erosion | 0 (no symptoms). 1 (mild), 2 (moderate), 3 (severe) |
| Dryness, scaling | 0 (no symptoms). 1 (mild), 2 (moderate), 3 (severe) |

TABLE A9
Changes in the skin damage symptoms of Nc/Nga mice with short
term atopic dermatitis by the topical application of Lipo-hPL

| Animal | | | | | Overall | |
|---|---|---|---|---|---|---|
| Treatment | Erythema | Edema | Erosion | Dryness | damage | S.D |
| No TNCB | 0 | 0 | 0 | 0 | 0 | 0 |
| treatment, | | | | | | |
| No Lipo-hPL | | | | | | |
| TNCB treatment; | 8 | 4 | 9 | 6 | 9.0 | 0.8 |
| No Lipo-hPL | | | | | | |
| TNCB treatment; | 3 | 0 | 3 | 3 | 3.0 | 0.3 |
| No Lipo-hPL, | | | | | | |
| but with | | | | | | |
| Tacrolimua | | | | | | |
| (FK506) | | | | | | |
| TNCB treatment; | 3 | 0 | 2 | 3 | 2.7 | 0.2 |
| Lipo-hPL | | | | | | |
| (20 μg/ml) | | | | | | |

TABLE A10
Changes in the skin damage symptoms of Nc/Nga mice with long
term atopic dermatitis by the topical application of Lipo-hPL

| Animal | | | | | Overall | |
|---|---|---|---|---|---|---|
| Treatment | Erythema | Edema | Erosion | Dryness | damage | S.D |
| No TNCB | 0 | 0 | 0 | 0 | 0 | 0 |
| treatment, | | | | | | |
| No Lipo-hPL | | | | | | |
| TNCB treatment; | 2 | 2 | 4.5 | 6 | 7.3 | 0.7 |
| No Lipo-hPL | | | | | | |
| TNCB treatment; | 1.5 | 1.5 | 3.5 | 3 | 4.8 | 0.7 |
| No Lipo-hPL, | | | | | | |
| but with | | | | | | |
| Tacrolimua | | | | | | |
| (FK506) | | | | | | |
| TNCB treatment; | 1 | 0 | 0.5 | 1.5 | 1.5 | 0.4 |
| Lipo-hPL | | | | | | |
| (20 μg/ml) | | | | | | |

TABLE A11
Formula 1. Shampoo

| Ingredient | | Weight % |
|---|---|---|
| A. | Water (aqua) | q.s. to 100.00% |
| | Sodium laureth sulfate | 20.00 |
| | Cocamide MEA | 2.00 |
| | Cocamidopropyl betaine | 7.00 |
| | Dimethicone copolyol | 2.00 |
| | PEG-6 caprylic capric glycerides | 1.00 |
| | Decyl glucoside | 3.00 |
| | Glycerin | 5.00 |
| | Preservatives | 0.30 |
| B. | Polysorbate 20 | 2.00 |
| | Fragrance (parfum) | 1.00 |
| C. | Lipo-hPL | 1.00 |

Procedure: Combine A and heat to 70° C. Begin to cool to 45° C. Mix until uniform. Add B and mix well. Cool to ambient temperature. Add C and mix until uniform.

TABLE A12
Formula 2. Emulsion (Lotion)

| Ingredient | | Weight % |
|---|---|---|
| A. | Mineral oil | 20.00 |
| | Dioctyldodecyl dodecanedioate | 2.00 |
| | Dimethicone | 1.00 |
| | Glyceryl stearate | 5.50 |
| | Cetyl esters | 1.00 |
| | Polysorbate 60 | 4.00 |
| | Cetyl alcohol | 1.00 |
| | Tocopheryl acetate | 0.10 |
| B. | Glycerin | 5.00 |
| | Water (aqua) | q.s. to 100.00% |
| | Preservatives | 0.50 |
| C. | Fragrance (parfum) | 0.10 |
| D. | Lipo-hPL | 2.00 |

Procedure: Heat A to 75° C. Separately, preblend B and heat to 75° C. and combine with A using rapid mixing. Cool to 45° C. and add the C. Cool to ambient temperature. Add D and mix until uniform.

TABLE A13
Formula 3. Liquid (Toner)

| Ingredient | | Weight % |
|---|---|---|
| A. | Water (aqua) | q.s. to 100.00% |
| B. | Glycerin | 8.00 |
| | Panthanol | 0.20 |
| | Preservatives | 0.30 |
| C. | Isoceteth-20 | 1.70 |
| | Fragrance (parfum) | 0.05 |
| D. | Lipo-hPL | 1.00 |

Procedure: Dissolve B in A in order. Add C and mix until homogeneous. Add D.

TABLE A14
Formula 4. Cream

| Ingredient | | Weight % |
|---|---|---|
| A. | Water (Aqua) | q.s. to 100.00% |
| B. | Hydroxyethylcellulose | 0.50 |
| C. | Glycerin | 5.00 |
| | Panthanol | 0.50 |
| | Preservatives | 0.50 |
| D. | Tocopheryl acetate | 0.10 |
| | Grape seed oil | 5.00 |
| | Squalane | 3.00 |
| | Caprylic/capric triglyceride | 6.00 |
| | Glyceryl stearate | 3.00 |
| | Polysorbate 60 | 1.00 |
| | Dimethicone | 3.00 |
| | Cetearyl alcohol | 1.00 |
| E. | Lipo-hPL | 2.00 |

Procedure: Hydrate B in A. Add C and heat to 75° C. Separately, preblend D and heat to 75° C. Add D to ABC under homogenization. Homogenize until emulsion forms. Cool to ambient temperature. Add D and mix until uniform.

TABLE A15
Formula 5. Essence (Gel)

| Ingredient | | Weight % |
|---|---|---|
| A. | Water (Aqua) | q.s. to 100.00% |
| | Carbomer | 0.40 |
| B. | Trehalose | 1 |
| | Polysorbate 60 | 0.1 |
| | Glycerin | 10 |
| | Preservatives | 0.5 |
| C. | Sodium hydroxide, to pH 6.3~6.7 | q.s |
| D | Lipo-hPL | 2.00 |

Procedure: Disperse the carbomer in the water to prepare A. Add B under stirring. Neutralize With C. Add D and mix until homogeneous.

What is claimed is:

1. A method for improving skin conditions of a human, which comprises topically administering to the skin of human a composition comprising an effective amount of human placental lactogen as an active ingredient, wherein the skin condition is selected from the group consisting of atopic dermatitis, wrinkles, age spots, acne, itching, poor skin elasticity, poor hair growth, skin aging and poor skin moisture; wherein the composition is applied to a normal skin surface that is not in direct contact with blood.

2. The method according to claim 1, wherein the human placental lactogen is encapsulated into a liposome.

3. The method according to claim 2, wherein the liposome is a nanoliposome.

4. The method according to claim 3, wherein the nanoliposome has a particle size of 50-250 nm.

5. The method according to claim 3, wherein the nanoliposome has a small unilamellar vesicle structure.

6. The method according to claim 3, wherein the human growth hormone encapsulated into the nanoliposome has the activity of 90-100% of an unencapsulated human placental lactogen.

7. The method according to claim 1, wherein the composition is a cosmetic or pharmaceutical composition.

8. A method for improving skin conditions of a human, which comprises topically administering to the skin of human a composition comprising an effective amount of human placental lactogen as an active ingredient, wherein the skin condition is selected from the group consisting of atopic dermatitis, contact dermatitis, skin damages caused by ultraviolet-light exposure, photoaging, UV or sunburn-induced pigmentation, wrinkles, age spots, acne, dryness, itching, hair-loss, actinic keratosis, eczema, psoriasis, sunburn, skin elasticity problems, and skin aging.


SEQUENCE LISTING (11 sequences)

SEQ ID NO: 1 - DNA, 36 bp, Artificial Sequence
  Description of Artificial Sequence Synthetic primer nucleotide sequence of Human Placental Lactogen (hPL 1U)
gggaattccatatggctccaggctcaaaccgttccc

SEQ ID NO: 2 - DNA, 34 bp, Artificial Sequence
  Description of Artificial Sequence Synthetic primer nucleotide sequence of Human Placental Lactogen (hPL 2D)
ataggtttcttcaaactcctggtaggtgtcaatg

SEQ ID NO: 3 - DNA, 34 bp, Artificial Sequence
  Description of Artificial Sequence Synthetic primer nucleotide sequence of Human Placental Lactogen (hPL 3U)
taccaggagtttgaagaaacctatatcccaaagg

SEQ ID NO: 4 - DNA, 33 bp, Artificial Sequence
  Description of Artificial Sequence Synthetic primer nucleotide sequence of Human Placental Lactogen (hPL 4D)
cagctctagattggatttctgttgcgtttcctc

SEQ ID NO: 5 - DNA, 33 bp, Artificial Sequence
  Description of Artificial Sequence Synthetic primer nucleotide sequence of Human Placental Lactogen (hPL 5U)
caacagaaatccaatctagagctgctccgcatc

SEQ ID NO: 6 - DNA, 31 bp, Artificial Sequence
  Description of Artificial Sequence Synthetic primer nucleotide sequence of Human Placental Lactogen (hPL 6D)
gtcttccagcctccccatcagcgtttggatg

SEQ ID NO: 7 - DNA, 30 bp, Artificial Sequence
  Description of Artificial Sequence Synthetic primer nucleotide sequence of Human Placental Lactogen (hPL 7U)
acgctgatggggaggctggaagacggcagc

SEQ ID NO: 8 - DNA, 28 bp, Artificial Sequence
  Description of Artificial Sequence Synthetic primer nucleotide sequence of Human Placental Lactogen (hPL 8D)
cggggtaccctagaagccacagctgccc

SEQ ID NO: 9 - DNA, 34 bp, Artificial Sequence
  Description of Artificial Sequence Synthetic primer nucleotide sequence of Human Placental Lactogen (hPL 9U)
gggaattccatatggtccaaaccgttcccttatc

SEQ ID NO: 10 - protein, 216 aa, Homo sapiens
APGSRTSLLLAFALLCLPWLQEAGAVQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 11 - DNA, 651 bp, Homo sapiens
gctccaggctcccggacgtccctgctcctggcttttgccctgctctgcctgccctggcttcaagaggctggtgccgtccaaaccgttcccttatccaggctttttgaccacgctatgctccaagcccatcgcgcgcaccagctggccattgacacctaccaggagtttgaagaaacctatatcccaaaggaccagaagtattcattcctgcatgactcccagacctccttctgcttctcagactctattccgacaccctccaacatggaggaaacgcaacagaaatccaatctagagctgctccgcatctccctgctgctcatcgagtcgtggctggagcccgtgcggttcctcaggagtatgttcgccaacaacctggtgtatgacacctcggacagcgatgactatcacctcctaaaggacctagaggaaggcatccaaacgctgatggggaggctggaagacggcagccgccggactgggcagatcctcaagcagacctacagcaagtttgacacaaactcgcacaaccatgacgcactgctcaagaactacgggctgctctactgcttcaggaaggacatggacaaggtcgagacattcctgcgcatggtgcagtgccgctctgtggagggcagctgtggcttctag